(19) United States
(12) Patent Application Publication
(10) Pub. No.: US 2006/0020023 A1
(43) Pub. Date: Jan. 26, 2006

(54) Racemization method

(75) Inventors: John Sauhoi Ng, Oak Park, CA (US); Joseph J. Wieczorek, Cary, IL (US)

Correspondence Address: WARNER-LAMBERT COMPANY, 2800 PLYMOUTH RD, ANN ARBOR, MI, 48105, US

(73) Assignee: PHARMACIA & UPJOHN COMPANY LLC

(21) Appl. No.: 11/187,768

(22) Filed: Jul. 23, 2005

Related U.S. Application Data
(60) Provisional application No. 60/590,515, filed on Jul. 23, 2004.

(51) Int. Cl. C07D 311/04; A61K 31/353

(52) U.S. Cl. 514/456; 549/403

(57) ABSTRACT

This invention relates to a method for racemizing enantiomers of a substituted 2-trifluoromethyl-2H-chromene-3-carboxylic acid or ester, a substituted 2-trifluoromethyl-1,2-dihydro-quinoline-3-carboxylic acid or ester, a substituted 2-trifluoromethyl-2H-thiochromene-3-carboxylic acid or ester, or a pharmaceutically acceptable salt of the acids or esters, using secondary amines, and optionally hydroxides, alkoxides, or sulfites at reaction mixture temperatures of from about 30° C. (i.e., above room temperature) to less than 300° C.

### CROSS-REFERENCE TO RELATED APPLICATIONS

This application claims priority from U.S. Provisional Patent Application No. 60/590,515 filed Jul. 23, 2004.

### FIELD OF THE INVENTION

This invention relates to a method for racemizing enantiomers of a substituted 2-trifluoromethyl-2H-chromene-3-carboxylic acid or ester, a substituted 2-trifluoromethyl-1,2-dihydro-quinoline-3-carboxylic acid or ester, a substituted 2-trifluoromethyl-2H-thiochromene-3-carboxylic acid or ester, or a pharmaceutically acceptable salt of the acids or esters, using secondary amines, and optionally hydroxides, alkoxides, or sulfites at reaction mixture temperatures of from about 30° C. (i.e., above room temperature) to less than 300° C.

### BACKGROUND OF THE INVENTION

Substituted 2-trifluoromethyl-2H-chromene-3-carboxylic acids and derivatives thereof are described in U.S. Pat. No. 6,034,256; 6,077,850; 6,218,427; or 6,271,253 or U.S. patent application Ser. Nos. 10/801,446 or 10/801,429. The derivatives thereof include compounds such as esters thereof, substituted 2-trifluoromethyl-1,2-dihydro-quinoline-3-carboxylic acids or esters, substituted 2-trifluoromethyl-2H-thiochromene-3-carboxylic acids or esters, and substituted 3-trifluoromethyl-3,4-dihydro-naphthalene-2-carboxylic acids or esters, and pharmaceutically acceptable salts thereof. The substituted 2-trifluoromethyl-2H-chromene-3-carboxylic acids and derivatives thereof each have a chiral center at the 2-position of the chromene, quinoline, or thiochromene and the 3-position of the 3,4-dihydro-naphthalene. The ring carbon atom of the chiral center is bonded to four functional groups. Two of these four functional groups are a hydrogen atom and a R¹ group as defined therein or a trifluoromethyl (“CF₃”) group. The other two of these four functional groups are the group X as defined below and the sp carbon atom at the 3-position of the chromene, quinoline, and thiochromene or the 2-position of the 3,4-dihydro-naphthalene.

The chiral substituted 2-trifluoromethyl-2H-chromene-3-carboxylic acids and derivatives thereof comprise enantiomers having either the (S)- or the (R)-configuration of the four functional groups that are bonded to the carbon atom of the chiral center. The (S)- and (R)-configurations represent the three-dimensional orientation of the four functional groups about the chiral center carbon atom. The enantiomers having either the enantiomers of these chiral compounds having either the (S)- or the (R)-configuration about the carbon atom of the chiral center bonded to the R¹ group or 2-trifluoromethyl group are referred to herein as (2S)- and (2R)-enantiomers, respectively, or the (3S)- and (3R)-enantiomers in the case of the 3,4-dihydro-naphthalene derivatives. The (2S)-enantiomer is the antipode (i.e., non-superimposable mirror image) of the (2R)-enantiomer and vice versa. The (3S)-enantiomer is the antipode of the (3R)-enantiomer and vice versa.

Generally, the (2S)-, (2R)-, (3S)- and (3R)-enantiomers of the substituted 2-trifluoromethyl-2H-chromene-3-carboxylic acids and derivatives thereof are physically and chemically identical to each other except for how they rotate plane-polarized light and how they interact with other chiral molecules such as each other and biological enzymes, receptors, and the like. The (2S)-, (2R)-, (3S)- and (3R)-enantiomers of the substituted 2-trifluoromethyl-2H-chromene-3-carboxylic acids and derivatives thereof are more potent inhibitors of the enzyme cyclooxygenase-2 (“COX-2”) than of the enzyme cyclooxygenase-1 (“COX-1”).

These enantiomers represent a new generation of “COX-2 inhibitors.” Typically for a particular compound, either the (2S)- or the (2R)-enantiomer (or the (3S)- or the (3R)-enantiomer in the case of 3,4-dihydro-naphthalene derivatives) exhibits (a) more potency for COX-2, (b) greater selectivity for COX-2- over COX-1, or (c) different metabolic profiles using liver microsome preparations than that for the other of the (2S)- and (2R)-enantiomers (or the (3S)- or the (3R)-enantiomers). Sometimes it is the (2S)-enantiomer (or (3S)-enantiomer) and other times it is the (2R)-enantiomer (or (3R)-enantiomer), depending upon the particular compound being considered, that has the more potent or selective inhibitory activity or superior metabolic profile. Depending upon the potency or selectivity inhibitory activity, metabolic profile, or other biological activities of the particular compound being considered, sometimes the (2S)-enantiomer (or (3S)-enantiomer) is preferred for drug development and other times the (2R)-enantiomer (or (3R)-enantiomer) is preferred.

The substituted 2-trifluoromethyl-2H-chromene-3-carboxylic acids and derivatives thereof typically are synthesized as mixtures (racemic or otherwise) of their enantiomers because a commercially better, direct enantioselective synthesis has not been devised yet. In order to be able to make multi-kilogram quantities of a particular enantiomer substituted 2-trifluoromethyl-2H-chromene-3-carboxylic acid, or derivative thereof, widely available as a pharmaceutical agent to patients in need of treatment with a COX-2 inhibitor, a mixture of the enantiomer and its antipode has been separated by enantioselective fractional crystallization with a chiral auxiliary and/or enantioselective multicolumn chromatography over chiral stationary phase (see “Enantioselective Separation Method, PC26168, filed concurrently herewith). The goal of these enantioselective purification methods is to ultimately produce the more desired enantiomer in high (preferably >99.0%) enantiomeric excess (“e.e.”), which is the relative percent of one enantiomer in excess of its antipode and ignoring any other impurities (e.g., a mixture containing 99.5% of an enantiomer and 0.5% of its antipode has an e.e. of 99.0% and a mixture containing 90% of an enantiomer and 10% of its antipode has an e.e. of 80%). However, the less desired enantiomer, the mass balance of which is 50% of a racemic compound, is left behind in a mother liquor or waste stream, respectively.

There is a particular need for a cost-effective method of converting a less desired (2S)- or (2R)-enantiomer of a substituted 2-trifluoromethyl-2H-chromene-3-carboxylic acid, or derivative thereof, (i.e., the chromene, quinoline, and thiochromene derivatives) to the more desired antipode, or an enriched mixture, including a racemic mixture, that contains relatively more of the desired antipode than was present before the conversion step. After purification, if needed, to remove any impurities, the mixture that has been relatively enriched in the more desired antipode will be suitable for one of the above-referenced enantioselective separation methods.

### SUMMARY OF THE INVENTION

This invention relates to a method for racemizing an enantiomer of a substituted 2-trifluoromethyl-2H-chromene-3-carboxylic acid or derivative thereof, other than a 3,4-dihydro-naphthalene-2-carboxylic acid, ester, or pharmaceutically acceptable salt thereof, or a mixture of the enantiomer and its antipode.

In one aspect, the invention is a method for converting a (2S)- or (2R)-enantiomer of a substituted 2-trifluoromethyl-2H-chromene-3-carboxylic acid, or derivative thereof, the method comprising the step of:
Heating at a temperature of from greater than 30° C. to less than 300° C. a reaction mixture containing, but not limited to, components (a), (b), and optionally (c):
   (a) A compound of formula R^{K}N(H)—C(H)(R^{Q})CH₂—R^{L};
   (b) A (2S)- or (2R)-enantiomer of a substituted 2-trifluoromethyl-2H-chromene-3-carboxylic acid or derivative thereof; or a non-racemic mixture having a major component which is a (2S)- or (2R)-enantiomer of the substituted 2-trifluoromethyl-2H-chromene-3-carboxylic acid or derivative thereof, and a minor component which is the antipode of the (2S)- or (2R)-enantiomer; and optionally
   (c) A compound of formula M¹OR^{Y} or M²(OR^{Y})₂;
to yield a mixture of the (2S)- and (2R)-enantiomers that has been relatively enriched in the antipode of the (2S)- or (2R)-enantiomer; wherein the mixture that has been relatively enriched in the antipode of the (2S)- or (2R)-enantiomer is characterized as having an enantiomeric excess of the (2S)- or (2R)-enantiomer that is less than 90% of the enantiomeric excess of component (b); wherein:
the temperature is the temperature of the reaction mixture;
M¹ is Na, K, or Cs;
M² is Mg, Zn, or Ca;
Each R^{Y} independently is hydrogen, C₁-C₄ alkyl, benzyl, or HSO₃⁻; or
Two R^{Y} are taken together to form SO₃⁻;
R^{K} independently is C₁-C₄ alkyl or benzyl;
R^{Q} is hydrogen, C₁-C₄ alkyl, phenyl, benzyl, 4-hydroxybenzyl, —(CH₂)_{q}NH₂, or —(CH₂)_{q}OH;
or R^{K} and R^{Q} are taken together to form a C₁-C₄ alkylene;
q is an integer of from 1 to 4;
R^{L} is OH or NH₂;
C₁-C₄ alkyl is an unsubstituted straight or branched chain hydrocarbon radical having from 1 to 4 carbon atoms;
C₁-C₄ alkylene is an unsubstituted straight or branched chain hydrocarbon diradical having from 1 to 4 carbon atoms; and wherein:
the substituted 2-trifluoromethyl-2H-chromene-3-carboxylic acid or derivative thereof, is a compound of Formulas I″, I′, I, or II 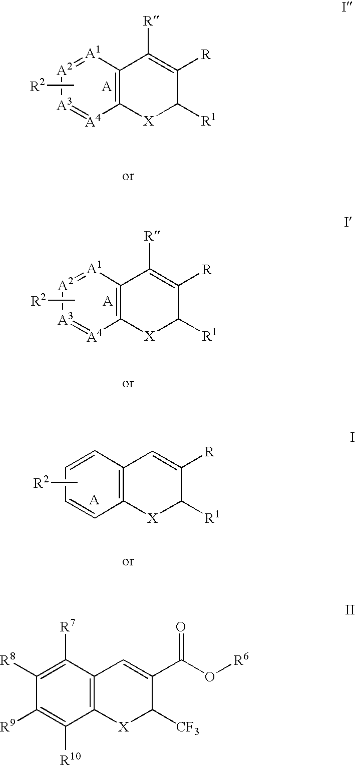
or a pharmaceutically acceptable salt thereof, wherein
for Formula I″:
wherein X is selected from O, S, and NR^{a};
wherein R^{a} is selected from hydrido, C₁-C₃-alkyl, (optionally substituted phenyl)-C₁-C₃-alkyl, acyl and carboxy-C₁-C₆-alkyl;
wherein R is selected from carboxyl, aminocarbonyl, C₁-C₆-alkylsulfonylaminocarbonyl and C₁-C₆-alkoxycarbonyl;
wherein R″ is selected from hydrido, phenyl, thienyl, C₁-C₆-alkyl and C₂-C₆-alkenyl;
wherein R¹ is CF₃;
wherein R² is one or more radicals independently selected from hydrido, halo, C₁-C₆-alkyl, C₂-C₆-alkenyl, C₂-C₆-alkynyl, halo-C₂-C₆-alkynyl, aryl-C₁-C₃-alkyl, aryl-C₂-C₆-alkynyl, aryl-C₂-C₆-alkenyl, C₁-C₆-alkoxy, methylenedioxy, C₁-C₆-alkylthio, C₁-C₆-alkylsulfinyl, aryloxy, arylthio, arylsulfinyl, heteroaryloxy, C₁-C₆-alkoxy-C₁-C₆-alkyl, aryl-C₁-C₆-alkyloxy, heteroaryl-C₁-C₆-alkyloxy, aryl-C₁-C₆-alkoxy-C₁-C₆-alkyl, C₁-C₆-haloalkyl, C₁-C₆-haloalkoxy, C₁-C₆-haloalkylthio, C₁-C₆-haloalkylsulfinyl, C₁-C₆-haloalkylsulfonyl, C₁-C₃-(haloalkyl-C₁-C₃-hydroxyalkyl, C₁-C₆-hydroxyalkyl, hydroxyimino-C₁-C₆-alkyl, C₁-C₆-alkylamino, arylamino, aryl-C₁-C₆-alkylamino, heteroarylamino, heteroaryl-C₁-C₆-alkylamino, nitro, cyano, amino, aminosulfonyl, C₁-C₆-alkylaminosulfonyl, arylaminosulfonyl, heteroarylaminosulfonyl, aryl-C₁-C₆-alkylaminosulfonyl, heteroaryl-C₁-C₆-alkylaminosulfonyl, heterocyclylsulfonyl, C₁-C₆-alkylsulfonyl, aryl-C₁-C₆-alkylsulfonyl, optionally substituted aryl, optionally substituted heteroaryl, aryl-C₁-C₆-alkylcarbonyl, heteroaryl-C₁-C₆-alkylcarbonyl, heteroarylcarbonyl, arylcarbonyl, aminocarbonyl, C₁-C₆-alkoxycarbonyl, formyl, C₁-C₆-haloalkylcarbonyl and C₁-C₆-alkylcarbonyl; and
wherein the A ring atoms A¹, A², A³ and A⁴ are independently selected from carbon and nitrogen with the proviso that at least two of A¹, A², A³ and A⁴ are carbon;
or wherein R² together with ring A forms a radical selected from naphthyl, quinolyl, isoquinolyl, quinolizinyl, quinoxalinyl and dibenzofuryl;
for Formula I′:
wherein X is selected from O, S, and NR^{a};
wherein R^{a} is selected from hydrido, C₁-C₃-alkyl, (optionally substituted phenyl)-C₁-C₃-alkyl, alkylsulfonyl, phenylsulfonyl, benzylsulfonyl, acyl and carboxy-C₁-C₆-alkyl;
wherein R is selected from carboxyl, aminocarbonyl, C₁-C₆-alkylsulfonylaminocarbonyl and C₁-C₆-alkoxycarbonyl;
wherein R″ is selected from hydrido, phenyl, thienyl, C₂-C₆-alkynyl and C₂-C₆-alkenyl;
wherein R¹ is CF₃;
wherein R² is one or more radicals independently selected from hydrido, halo, C₁-C₆-alkyl, C₂-C₆-alkenyl, C₂-C₆-alkynyl, halo-C₂-C₆-alkynyl, aryl-C₁-C₃-alkyl, aryl-C₂-C₆-alkynyl, aryl-C₂-C₆-alkenyl, C₁-C₆-alkoxy, methylenedioxy, C₁-C₆-alkylthio, C₁-C₆-alkylsulfinyl, —O(CF₂)₂O—, aryloxy, arylthio, arylsulfinyl, heteroaryloxy, C₁-C₆-alkoxy-C₁-C₆-alkyl, aryl-C₁-C₆-alkyloxy, heteroaryl-C₁-C₆-alkyloxy, aryl-C₁-C₆-alkoxy-C₁-C₆-alkyl, C₁-C₆-haloalkyl, C₁-C₆-haloalkoxy, C₁-C₆-haloalkylthio, C₁-C₆-haloalkylsulfinyl, C₁-C₆-haloalkylsulfonyl, C₁-C₃-(haloalkyl-C₁-C₃-hydroxyalkyl, C₁-C₆-hydroxyalkyl, hydroxyimino-C₁-C₆-alkyl, C₁-C₆-alkylamino, arylamino, aryl-C₁-C₆-alkylamino, heteroarylamino, heteroaryl-C₁-C₆-alkylamino, nitro, cyano, amino, aminosulfonyl, C₁-C₆-alkylaminosulfonyl, arylaminosulfonyl, heteroarylaminosulfonyl, aryl-C₁-C₆-alkylaminosulfonyl, heteroaryl-C₁-C₆-alkylaminosulfonyl, heterocyclylsulfonyl, C₁-C₆-alkylsulfonyl, aryl-C₁-C₆-alkylsulfonyl, optionally substituted aryl, optionally substituted heteroaryl, aryl-C₁-C₆-alkylcarbonyl, heteroaryl-C₁-C₆-alkylcarbonyl, heteroarylcarbonyl, arylcarbonyl, aminocarbonyl, C₁-C₆-alkoxycarbonyl, formyl, C₁-C₆-haloalkylcarbonyl and C₁-C₆-alkylcarbonyl; and
wherein the A ring atoms A¹, A², A³ and A⁴ are independently selected from carbon and nitrogen with the proviso that at least two of A¹, A², A³ and A⁴ are carbon;
or wherein R² together with ring A forms a radical selected from naphthyl, quinolyl, isoquinolyl, quinolizinyl, quinoxalinyl and dibenzofuryl;
for Formula I:
wherein X is selected from O or S or NR^{a};
wherein R^{a} is alkyl;
wherein R is selected from carboxyl, aminocarbonyl, alkylsulfonylaminocarbonyl and alkoxycarbonyl;
wherein R¹ is CF₃; and
wherein R² is one or more radicals selected from hydrido, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, optionally substituted aryl, optionally substituted heteroaryl, aralkylcarbonyl, heteroarylcarbonyl, arylcarbonyl, aminocarbonyl, and alkylcarbonyl;
or wherein R² together with ring A forms a naphthyl radical;
for Formula II:
wherein X is selected from O, S, and NH;
wherein R⁶ is H or alkyl; and
wherein R⁷, R⁹, R⁹, and R¹⁰ independently are selected from H, alkenyl, alkoxy, alkoxyalkyl, alkoxycarbonylalkyl, alkyl, alkylamino, alkylcarbonyl, alkylheteroaryl, alkylsulfonylalkyl, alkylthio, alkynyl, aminocarbonylalkyl, aryl, arylalkenyl, arylalkoxy, arylalkyl, arylalkylamino, arylalkynyl, arylcarbonyl, aryloxy, cyano, dialkylamino, halo, haloalkoxy, haloalkyl, heteroaryl, heteroarylalkoxy, heteroarylcarbonyl, hydroxy and hydroxyalkyl; wherein each of aryl, wherever it occurs, is independently substituted with one to five substituents selected from the group consisting of alkyl, alkoxy, alkylamino, cyano, halo, haloalkyl, hydroxy, and nitro.

Another aspect of this invention is any one of the above or below methods for converting, wherein the component (b) is a (2S)- or (2R)-enantiomer of a compound of Formula I″, I′, I, or II wherein X is O, or a non-racemic mixture thereof.

Another aspect of this invention is any one of the above or below methods for converting, wherein the component (b) is a (2S)- or (2R)-enantiomer of a compound of Formula II wherein X is O and R⁶ is H, or a non-racemic mixture thereof.

Another aspect of this invention is any one of the above or below methods for converting, wherein the component (b) is (R)-6-chloro-7-tert-butyl-2-trifluoromethyl-2H-chromene-3-carboxylic acid; or the component (b) is a non-racemic mixture having a major component which is (R)-6-chloro-7-tert-butyl-2-trifluoromethyl-2H-chromene-3-carboxylic acid and a minor component which is the antipode (S)-6-chloro-7-tert-butyl-2-trifluoromethyl-2H-chromene-3-carboxylic acid.

Another aspect of this invention is any one of the above or below methods for converting, wherein the component (b) is:
(R)-6-chloro-8-methyl-2-trifluoromethyl-2H-chromene-3-carboxylic acid;
(R)-6-chloro-5,7-dimethyl-2-trifluoromethyl-2H-chromene-3-carboxylic acid;
(R)-6,8-dimethyl-2-trifluoromethyl-2H-chromene-3-carboxylic acid; or
(R)-8-ethyl-6-trifluoromethoxy-2-trifluoromethyl-2H-chromene-3-carboxylic acid; or
the component (b) is a non-racemic mixture having a major component which is:
(R)-6-chloro-8-methyl-2-trifluoromethyl-2H-chromene-3-carboxylic acid;
(R)-6-chloro-5,7-dimethyl-2-trifluoromethyl-2H-chromene-3-carboxylic acid;
(R)-6,8-dimethyl-2-trifluoromethyl-2H-chromene-3-carboxylic acid; or
(R)-8-ethyl-6-trifluoromethoxy-2-trifluoromethyl-2H-chromene-3-carboxylic acid; and
a minor component which is the antipode:
(S)-6-chloro-8-methyl-2-trifluoromethyl-2H-chromene-3-carboxylic acid;
(S)-6-chloro-5,7-dimethyl-2-trifluoromethyl-2H-chromene-3-carboxylic acid;
(S)-6,8-dimethyl-2-trifluoromethyl-2H-chromene-3-carboxylic acid; or
(S)-8-ethyl-6-trifluoromethoxy-2-trifluoromethyl-2H-chromene-3-carboxylic acid, respectively.

Another aspect of this invention is any one of the above or below methods for converting, wherein the reaction mixture further contains a means for enantioselective fractional crystallization of the antipode of the (2S)- or (2R)-enantiomer.

Another aspect of this invention is any one of the above or below methods for converting, wherein the component (b) is:
(R)-6-chloro-7-tert-butyl-2-trifluoromethyl-2H-chromene-3-carboxylic acid (+)-cinchonine salt; or
(R)-6-chloro-7-tert-butyl-2-trifluoromethyl-2H-chromene-3-carboxylic acid D-phenylalaninol salt; or
the component (b) is a non-racemic mixture having a major component which is:
(R)-6-chloro-7-tert-butyl-2-trifluoromethyl-2H-chromene-3-carboxylic acid (+)-cinchonine salt; or
(R)-6-chloro-7-tert-butyl-2-trifluoromethyl-2H-chromene-3-carboxylic acid D-phenylalaninol salt; and
a minor component which is the antipode:
(S)-6-chloro-7-tert-butyl-2-trifluoromethyl-2H-chromene-3-carboxylic acid (+)-cinchonine salt; or
(S)-6-chloro-7-tert-butyl-2-trifluoromethyl-2H-chromene-3-carboxylic acid D-phenylalaninol salt, respectively.

Another aspect of this invention is any one of the above or below methods for converting, wherein the component (b) is:
(R)-6-chloro-8-methyl-2-trifluoromethyl-2H-chromene-3-carboxylic acid
(R)-(+)-N-benzyl-α-methylbenzylamine salt;
(R)-6-chloro-5,7-dimethyl-2-trifluoromethyl-2H-chromene-3-carboxylic acid (−)-cinchonine salt;
(R)-6,8-dimethyl-2-trifluoromethyl-2H-chromene-3-carboxylic acid (R)-(+)-N-benzyl-α-methylbenzylamine salt; or
(R)-8-ethyl-6-trifluoromethoxy-2-trifluoromethyl-2H-chromene-3-carboxylic acid (R)-(+)-N-benzyl-α-methylbenzylamine salt; or the component (b) is a non-racemic mixture having a major component which is:
(R)-6-chloro-8-methyl-2-trifluoromethyl-2H-chromene-3-carboxylic acid,
(R)-(+)-N-benzyl-α-methylbenzylamine salt;
(R)-6-chloro-5,7-dimethyl-2-trifluoromethyl-2H-chromene-3-carboxylic acid, (−)-cinchonine salt;
(R)-6,8-dimethyl-2-trifluoromethyl-2H-chromene-3-carboxylic acid, (R)-(+)-N-benzyl-α-methylbenzylamine salt; or
(R)-8-ethyl-6-trifluoromethoxy-2-trifluoromethyl-2H-chromene-3-carboxylic acid, (R)-(+)-N-benzyl-α-methylbenzylamine salt; and a minor component which is the antipode:
(S)-6-chloro-8-methyl-2-trifluoromethyl-2H-chromene-3-carboxylic acid,
(R)-(+)-N-benzyl-α-methylbenzylamine salt;
(S)-6-chloro-5,7-dimethyl-2-trifluoromethyl-2H-chromene-3-carboxylic acid, (−)-cinchonine salt;
(S)-6,8-dimethyl-2-trifluoromethyl-2H-chromene-3-carboxylic acid, (R)-(+)-N-benzyl-α-methylbenzylamine salt; or
(S)-8-ethyl-6-trifluoromethoxy-2-trifluoromethyl-2H-chromene-3-carboxylic acid, (R)-(+)-N-benzyl-α-methylbenzylamine salt, respectively.

Another aspect of this invention is any one of the above or below methods for converting, wherein R^{L} is OH.

Another aspect of this invention is any one of the above or below methods for converting, wherein each R^{K} independently is C₁-C₄ alkyl.

Another aspect of this invention is any one of the above or below methods for converting, wherein R^{Q} is hydrogen.

Another aspect of this invention is any one of the above or below methods for converting, wherein the compound of formula M¹OR^{Y} is a compound of formula NaOR^{Y}, wherein R^{Y} is as defined above.

Another aspect of this invention is any one of the above or below methods for converting, wherein each R^{Y} is hydrogen.

Another aspect of this invention is any one of the above or below methods for converting, wherein R^{Y} is HSO₃⁻ or two R^{Y} are taken together to form SO₃⁻.

### DETAILED DESCRIPTION OF THE INVENTION

The invention provides a method for converting a (2S)- or (2R)-enantiomer of a substituted 2-trifluoromethyl-2H-chromene-3-carboxylic acid, or derivative thereof, the method comprising the step of:
Heating at a temperature of from about 30° C. and less than 300° C. a reaction mixture containing, but not limited to, components (a), (b), and optionally (c):
   (a) A compound of formula R^{K}N(H)—C(H)(R^{Q})CH₂—R^{L},
   (b) A (2S)- or (2R)-enantiomer of a substituted 2-trifluoromethyl-2H-chromene-3-carboxylic acid or derivative thereof; or a non-racemic mixture having a major component which is a (2S)- or (2R)-enantiomer of the substituted 2-trifluoromethyl-2H-chromene-3-carboxylic acid or derivative thereof, and a minor component which is the antipode of the (2S)- or (2R)-enantiomer; and optionally
   (c) A compound of formula M¹OR^{Y} or M²(OR^{Y})₂;
to yield a mixture of the (2S)- and (2R)-enantiomers that has been relatively enriched in the antipode of the (2S)- or (2R)-enantiomer; wherein the mixture that has been relatively enriched in the antipode of the (2S)- or (2R)-enantiomer is characterized as having an enantiomeric excess of the (2S)- or (2R)-enantiomer that is less than 90% of the enantiomeric excess of component (b); wherein the temperature of the reaction mixture, the compound of formula R^{K}N(H)—C(H)(R^{Q})CH₂—R^{L} and the compound of formula M¹OR^{Y} or M² (OR^{Y})₂ are as defined above and wherein the substituted 2-trifluoromethyl-2H-chromene-3-carboxylic acid or derivative thereof, or a pharmaceutically acceptable salt thereof, is a compound of Formulas I″, I′, I, or II as defined above. Additional aspects of the present invention are described above and below.

A derivative of a substituted 2-trifluoromethyl-2H-chromene-3-carboxylic acid includes a substituted 2-trifluoromethyl-2H-chromene-3-carboxylic ester, a substituted 2-trifluoromethyl-1,2-dihydro-quinoline-3-carboxylic acid and ester, and a substituted 2-trifluoromethyl-2H-thiochromene-3-carboxylic acid and ester, and a pharmaceutically acceptable salt thereof.

Substituted 3-trifluoromethyl-3,4-dihydro-naphthalene-2-carboxylic acids and esters, and pharmaceutically acceptable salts thereof, are excluded from the present invention method.

An “acid derivative” of a substituted 2-trifluoromethyl-2H-chromene-3-carboxylic acid includes a substituted 2-trifluoromethyl-1,2-dihydro-quinoline-3-carboxylic acid, and a substituted 2-trifluoromethyl-2H-thiochromene-3-carboxylic acid.

An “ester derivative” of a substituted 2-trifluoromethyl-2H-chromene-3-carboxylic acid includes a substituted 2-trifluoromethyl-2H-chromene-3-carboxylic ester, a substituted 2-trifluoromethyl-1,2-dihydro-quinoline-3-carboxylic ester, and a substituted 2-trifluoromethyl-2H-thiochromene-3-carboxylic ester.

A “pharmaceutically acceptable salt thereof” means a pharmaceutically acceptable salt of a substituted 2-trifluoromethyl-2H-chromene-3-carboxylic acid or a salt of a derivative of the substituted 2-trifluoromethyl-2H-chromene-3-carboxylic acid.

The terms “pharmaceutically-acceptable salts” and “pharmaceutically acceptable salts” are synonymous. Both terms embrace salts commonly used to form alkali metal salts and to form addition salts of free acids or free bases.

Many substituted 2-trifluoromethyl-2H-chromene-3-carboxylic acids, and esters having a basic nitrogen atom, are capable of further forming pharmaceutically acceptable salts, including, but not limited to, base addition salts and acid addition salts, respectively. Suitable pharmaceutically-acceptable acid addition salts of compounds of Formulas I″, I′, I, and II may be prepared from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, araliphatic, heterocyclic, carboxylic and sulfonic classes of organic acids, example of which are formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, salicyclic, salicyclic, 4-hydroxybenzoic, phenylacetic, mandelic, embonic (pamoic), methanesulfonic, ethanesulfonic, benzenesulfonic, pantothenic, 2-hydroxyethanesulfonic, toluenesulfonic, sulfanilic, cyclohexylaminosulfonic, stearic, algenic, β-hydroxybutyric, salicyclic, galactaric and galacturonic acid. Suitable pharmaceutically-acceptable base addition salts of compounds of Formulas I″, I′, I, and II include metallic salts, such as salts made from aluminum, calcium, lithium, magnesium, potassium, sodium and zinc, or salts made from organic bases including primary, secondary and tertiary amines, substituted amines including cyclic amines, such as caffeine, arginine, diethylamine, N-ethyl piperidine, histidine, glucamine, isopropylamine, lysine, morpholine, N-ethyl morpholine, piperazine, piperidine, triethylamine, trimethylamine. All of these salts may be prepared by conventional means from the corresponding compound of the invention by reacting, for example, the appropriate acid or base with the compound of Formulas I″, I′, I, and II.

For purposes herein, a substituted 2-trifluoromethyl-2H-chromene-3-carboxylic acid or ester, or a pharmaceutically acceptable salt thereof (i.e., a compound of Formulas I″, I′, I, or II wherein X is O), a substituted 2-trifluoromethyl-1,2-dihydro-quinoline-3-carboxylic acid or ester, or a pharmaceutically acceptable salt thereof (i.e., a compound of Formulas I″, I′, or I, wherein X is NR^{a} or a compound of Formula II wherein is NH), and a substituted 2-trifluoromethyl-2H-thiochromene-3-carboxylic acid or ester, or a pharmaceutically acceptable salt thereof (i.e., a compound of Formulas I″, I′, I, or II wherein X is S), will have the ring numbering scheme illustrated below: 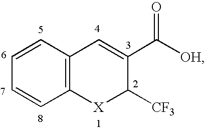
wherein X is O, S, NH, or NR^{a}.

A 2H-chromene-3-carboxylic acid (X is O) may also be known as a 2H-1-benzopyran-3-carboxylic acid.

For the compound of formula R^{K}N(H)—C(H)(R^{Q})CH₂—R^{L} or the compound of formula M¹OR^{Y} or M²(OR^{Y})₂, the following terms are defined:

The term “C₁-C₄ alkyl” means a straight or branched hydrocarbon radical having from 1 to 4 carbon atoms. Illustrative examples of a C₁-C₄ alkyl include methyl, ethyl, 1-propyl, 2-propyl, and 1,1-dimethylethyl (i.e., tertiary-butyl).

The term “C₁-C₄ alkylene” means a straight or branched hydrocarbon diradical having from 1 to 4 carbon atoms. Illustrative examples of a C₁-C₄ alkylene include CH₂, CH₂CH₂, CH₂CH₂CH₂, CH(CH₃)CH₂, CH₂CH₂CH₂CH₂, and C(CH₃)CH₂.

For a compound of Formulas I″, I′, and I, the following terms are defined:

The term “hydrido” denotes a single hydrogen atom (H). This hydrido radical may be attached, for example, to an oxygen atom to form a hydroxyl radical or two hydrido radicals may be attached to a carbon atom to form a methylene (—CH₂—) radical.

Where the term “alkyl” is used, either alone or within other terms such as “haloalkyl” and “alkylsulfonyl”, it embraces linear or branched radicals having one to about twenty carbon atoms or, preferably, one to about twelve carbon atoms. More preferred alkyl radicals are “lower alkyl” radicals having one to about six carbon atoms. Examples of such radicals include methyl, ethyl, n-propyl, isopropyl, n-butyl, isobutyl, sec-butyl, tert-butyl, pentyl, iso-amyl, hexyl and the like. Even more preferred are lower alkyl radicals having one to three carbon atoms.

The term “alkenyl” embraces linear or branched radicals having at least one carbon-carbon double bond of two to about twenty carbon atoms or, preferably, two to about twelve carbon atoms. More preferred alkenyl radicals are “lower alkenyl” radicals having two to about six carbon atoms. Examples of alkenyl radicals include ethenyl, propenyl, allyl, propenyl, butenyl and 4-methylbutenyl.

The term “alkynyl” denotes linear or branched radicals having two to about twenty carbon atoms or, preferably, two to about twelve carbon atoms. More preferred alkynyl radicals are “lower alkynyl” radicals having two to about ten carbon atoms. Most preferred are lower alkynyl radicals having two to about six carbon atoms. Examples of such radicals include propargyl, butynyl, and the like.

The terms “alkenyl” and “lower alkenyl”, embrace radicals having “cis” and “trans” orientations, or alternatively, “E” and “Z” orientations.

The term “halo” means halogens such as fluorine, chlorine, bromine or iodine atoms.

The term “haloalkyl” embraces radicals wherein any one or more of the alkyl carbon atoms is substituted with halo as defined above. Specifically embraced are monohaloalkyl, dihaloalkyl and polyhaloalkyl radicals. A monohaloalkyl radical, for one example, may have either an iodo, bromo, chloro fluoro atom within the radical. Dihalo and polyhaloalkyl radicals may have two or more of the same halo atoms or a combination of different halo radicals.

“Lower haloalkyl” embraces radicals having 1-6 carbon atoms. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl.

“Perfluoroalkyl” means alkyl radicals having all hydrogen atoms replaced with fluoro atoms. Examples include trifluoromethyl and pentafluoroethyl.

The term “hydroxyalkyl” embraces linear or branched alkyl radicals having one to about ten carbon atoms any one of which may be substituted with one or more hydroxyl radicals. More preferred hydroxyalkyl radicals are “lower hydroxyalkyl” radicals having one to six carbon atoms and one or more hydroxyl radicals. Examples of such radicals include hydroxymethyl, hydroxyethyl, hydroxypropyl, hydroxybutyl and hydroxyhexyl. Even more preferred are lower hydroxyalkyl radicals having one to three carbon atoms.

The term “cyanoalkyl” embraces linear or branched alkyl radicals having one to about ten carbon atoms any one of which may be substituted with one cyano radicals. More preferred cyanoalkyl radicals are “lower cyanoalkyl” radicals having one to six carbon atoms and one cyano radical. Even more preferred are lower cyanoalkyl radicals having one to three carbon atoms. Examples of such radicals include cyanomethyl.

The terms “alkoxy” embrace linear or branched oxy-containing radicals each having alkyl portions of one to about ten carbon atoms. More preferred alkoxy radicals are “lower alkoxy” radicals having one to six carbon atoms. Examples of such radicals include methoxy, ethoxy, propoxy, butoxy and tert-butoxy. Even more preferred are lower alkoxy radicals having one to three carbon atoms. The “alkoxy” radicals may be further substituted with one or more halo atoms, such as fluoro, chloro or bromo, to provide “haloalkoxy” radicals. Even more preferred are lower haloalkoxy radicals having one to three carbon atoms. Examples of such radicals include fluoromethoxy, chloromethoxy, trifluoromethoxy, trifluoroethoxy, fluoroethoxy and fluoropropoxy.

The term “aryl”, alone or in combination in other terms (e.g., aryl-C₁-C₃ alkyl), means a carbocyclic aromatic system containing one or two rings wherein such rings may be attached together in a pendent manner or may be fused. The term “aryl” embraces aromatic radicals such as phenyl, naphthyl, tetrahydronaphthyl, indane and biphenyl. More preferred aryl is phenyl. The “aryl” group may have 1 to 3 substituents such as lower alkyl, hydroxy, halo, haloalkyl, nitro, cyano, alkoxy and lower alkylamino.

The term “heterocyclyl” embraces saturated, partially saturated and unsaturated heteroatom-containing ring-shaped radicals, where the heteroatoms may be selected from nitrogen, sulfur and oxygen. Examples of saturated heterocyclic radicals include saturated 3 to 6-membered heteromonocylic group containing 1 to 4 nitrogen atoms [e.g. pyrrolidinyl, imidazolidinyl, piperidino, piperazinyl]; saturated 3 to 6-membered heteromonocyclic group containing 1 to 2 oxygen atoms and 1 to 3 nitrogen atoms [e.g. morpholinyl]; saturated 3 to 6-membered heteromonocyclic group containing 1 to 2 sulfur atoms and 1 to 3 nitrogen atoms [e.g., thiazolidinyl]. Examples of partially saturated heterocyclyl radicals include dihydrothiophene, dihydropyran, dihydrofuran and dihydrothiazole. Examples of unsaturated heterocyclic radicals, also termed “heteroaryl” radicals, include unsaturated 5 to 6 membered heteromonocyclyl group containing 1 to 4 nitrogen atoms, for example, pyrrolyl, pyrrolinyl, imidazolyl, pyrazolyl, 2-pyridyl, 3-pyridyl, 4-pyridyl, pyrimidyl, pyrazinyl, pyridazinyl, triazolyl [e.g., 4H-1,2,4-triazolyl, 1H-1,2,3-triazolyl, 2H-1,2,3-triazolyl]; unsaturated condensed heterocyclic group containing 1 to 5 nitrogen atoms, for example, indolyl, isoindolyl, indolizinyl, benzimidazolyl, quinolyl, isoquinolyl, indazolyl, benzotriazolyl, tetrazolopyridazinyl [e.g., tetrazolo[1,5-b]pyridazinyl]; unsaturated 3 to 6-membered heteromonocyclic group containing an oxygen atom, for example, pyranyl, 2-furyl, 3-furyl, etc.; unsaturated 5 to 6-membered heteromonocyclic group containing a sulfur atom, for example, 2-thienyl, 3-thienyl, etc.; unsaturated 5- to 6-membered heteromonocyclic group containing 1 to 2 oxygen atoms and 1 to 3 nitrogen atoms, for example, oxazolyl, isoxazolyl, oxadiazolyl [e.g., 1,2,4-oxadiazolyl, 1,3,4-oxadiazolyl, 1,2,5-oxadiazolyl]; unsaturated condensed heterocyclic group containing 1 to 2 oxygen atoms and 1 to 3 nitrogen atoms [e.g. benzoxazolyl, benzoxadiazolyl]; unsaturated 5 to 6-membered heteromonocyclic group containing 1 to 2 sulfur atoms and 1 to 3 nitrogen atoms, for example, thiazolyl, thiadiazolyl [e.g., 1,2,4-thiadiazolyl, 1,3,4-thiadiazolyl, 1,2,5-thiadiazolyl]; unsaturated condensed heterocyclic group containing 1 to 2 sulfur atoms and 1 to 3 nitrogen atoms [e.g., benzothiazolyl, benzothiadiazolyl] and the like. The term also embraces radicals where heterocyclic radicals are fused with aryl radicals. Examples of such fused bicyclic radicals include benzofuran, benzothiophene, and the like. The “heterocyclyl” group may have 1 to 3 substituents such as lower alkyl, hydroxy, oxo, amino and lower alkylamino. Preferred heterocyclic radicals include five to ten membered fused or unfused radicals. More preferred examples of heteroaryl radicals include benzofuryl, 2,3-dihydrobenzofuryl, benzothienyl, indolyl, dihydroindolyl, chromanyl, benzopyran, thiochromanyl, benzothiopyran, benzodioxolyl, benzodioxanyl, pyridyl, thienyl, thiazolyl, oxazolyl, furyl, and pyrazinyl. Even more preferred heteroaryl radicals are 5- or 6-membered heteroaryl, containing one or two heteroatoms selected from sulfur nitrogen and oxygen, selected from thienyl, furanyl, pyrrolyl, thiazolyl, oxazolyl, imidazolyl, pyrazolyl, isoxazolyl, isothiazolyl, pyridyl, piperidinyl and pyrazinyl.

The term “sulfonyl”, whether used alone or linked to other terms such as alkylsulfonyl, denotes respectively divalent radicals —SO₂—.

“Alkylsulfonyl” embraces alkyl radicals attached to a sulfonyl radical, where alkyl is defined as above. More preferred alkylsulfonyl radicals are “lower alkylsulfonyl” radicals having one to six carbon atoms. Even more preferred are lower alkylsulfonyl radicals having one to three carbon atoms. Examples of such lower alkylsulfonyl radicals include methylsulfonyl, ethylsulfonyl and propylsulfonyl.

“Haloalkylsulfonyl” embraces haloalkyl radicals attached to a sulfonyl radical, where haloalkyl is defined as above. More preferred haloalkylsulfonyl radicals are “lower haloalkylsulfonyl” radicals having one to six carbon atoms. Even more preferred are lower haloalkylsulfonyl radicals having one to three carbon atoms. Examples of such lower haloalkylsulfonyl radicals include trifluoromethylsulfonyl.

The term “arylalkylsulfonyl” embraces aryl radicals as defined above, attached to an alkylsulfonyl radical. Examples of such radicals include benzylsulfonyl and phenylethylsulfonyl.

The term “heterocyclosulfonyl” embraces heterocyclo radicals as defined above, attached to a sulfonyl radical. More preferred heterocyclosulfonyl radicals contain 5-7 membered heterocyclo radicals containing one or two heteroatoms. Examples of such radicals include tetrahydropyrrolylsulfonyl morpholinylsulfonyl and azepinylsulfonyl.

The terms “sulfamyl,” “aminosulfonyl” and “sulfonamidyl,” whether alone or used with terms such as “N-alkylaminosulfonyl”, “N-arylaminosulfonyl”, “N,N-dialkylaminosulfonyl” and “N-alkyl-N-arylaminosulfonyl”, denotes a sulfonyl radical substituted with an amine radical, forming a sulfonamide (—SO₂NH₂).

The term “alkylaminosulfonyl” includes “N-alkylaminosulfonyl” and “N,N-dialkylaminosulfonyl” where sulfamyl radicals are substituted, respectively, with one alkyl radical, or two alkyl radicals. More preferred alkylaminosulfonyl radicals are “lower alkylaminosulfonyl” radicals having one to six carbon atoms. Even more preferred are lower alkylaminosulfonyl radicals having one to three carbon atoms. Examples of such lower alkylaminosulfonyl radicals include N-methylaminosulfonyl, N-ethylaminosulfonyl and N-methyl-N-ethylaminosulfonyl.

The terms “N-arylaminosulfonyl” and “N-alkyl-N-arylaminosulfonyl” denote sulfamyl radicals substituted, respectively, with one aryl radical, or one alkyl and one aryl radical. More preferred N-alkyl-N-arylaminosulfonyl radicals are “lower N-alkyl-N-arylsulfonyl” radicals having alkyl radicals of one to six carbon atoms. Even more preferred are lower N-alkyl-N-arylsulfonyl radicals having one to three carbon atoms. Examples of such lower N-alkyl-N-aryl-aminosulfonyl radicals include N-methyl-N-phenylaminosulfonyl and N-ethyl-N-phenylaminosulfonyl. Examples of such N-aryl-aminosulfonyl radicals include N-phenylaminosulfonyl.

The term “arylalkylaminosulfonyl” embraces aralkyl radicals as described above, attached to an aminosulfonyl radical. More preferred are lower arylalkylaminosulfonyl radicals having one to three carbon atoms.

The term “heterocyclylaminosulfonyl” embraces heterocyclyl radicals as described above, attached to an aminosulfonyl radical.

The terms “carboxy” or “carboxyl”, whether used alone or with other terms, such as “carboxyalkyl”, denotes —CO₂H.

The term “carboxyalkyl” embraces radicals having a carboxy radical as defined above, attached to an alkyl radical.

The term “carbonyl”, whether used alone or with other terms, such as “alkylcarbonyl”, denotes —(C═O)—.

The term “acyl” denotes a radical provided by the residue after removal of hydroxyl from an organic acid. Examples of such acyl radicals include alkanoyl and aroyl radicals. Examples of such lower alkanoyl radicals include formyl, acetyl, propionyl, butyryl, isobutyryl, valeryl, isovaleryl, pivaloyl, hexanoyl, trifluoroacetyl.

The term “aroyl” embraces aryl radicals with a carbonyl radical as defined above. Examples of aroyl include benzoyl, naphthoyl, and the like and the aryl in the aroyl may be additionally substituted.

The term “alkylcarbonyl” embraces radicals having a carbonyl radical substituted with an alkyl radical. More preferred alkylcarbonyl radicals are “lower alkylcarbonyl” radicals having one to six carbon atoms. Even more preferred are lower alkylcarbonyl radicals having one to three carbon atoms. Examples of such radicals include methylcarbonyl and ethylcarbonyl.

The term “haloalkylcarbonyl” embraces radicals having a carbonyl radical substituted with a haloalkyl radical. More preferred haloalkylcarbonyl radicals are “lower haloalkylcarbonyl” radicals having one to six carbon atoms. Even more preferred are lower haloalkylcarbonyl radicals having one to three carbon atoms. Examples of such radicals include trifluoromethylcarbonyl.

The term “arylcarbonyl” embraces radicals having a carbonyl radical substituted with an aryl radical. More preferred arylcarbonyl radicals include phenylcarbonyl.

The term “heteroarylcarbonyl” embraces radicals having a carbonyl radical substituted with a heteroaryl radical. Even more preferred are 5- or 6-membered heteroarylcarbonyl radicals.

The term “arylalkylcarbonyl” embraces radicals having a carbonyl radical substituted with an arylalkyl radical. More preferred radicals are phenyl-C₁-C₃-alkylcarbonyl, including benzylcarbonyl.

The term “heteroarylalkylcarbonyl” embraces radicals having a carbonyl radical substituted with a heteroarylalkyl radical. Even more preferred are lower heteroarylalkylcarbonyl radicals having 5-6-membered heteroaryl radicals attached to alkyl portions having one to three carbon atoms.

The term “alkoxycarbonyl” means a radical containing an alkoxy radical, as defined above, attached via an oxygen atom to a carbonyl radical. Preferably, “lower alkoxycarbonyl” embraces alkoxy radicals having one to six carbon atoms. Examples of such “lower alkoxycarbonyl” ester radicals include substituted or unsubstituted methoxycarbonyl, ethoxycarbonyl, propoxycarbonyl, butoxycarbonyl and hexyloxycarbonyl. Even more preferred are lower alkoxycarbonyl radicals having alkoxy portions of one to three carbon atoms.

The term “aminocarbonyl” when used by itself or with other terms such as “aminocarbonylalkyl”, “N-alkylaminocarbonyl”, “N-arylaminocarbonyl”, “N,N-dialkylaminocarbonyl”, “N-alkyl-N-arylaminocarbonyl”, “N-alkyl-N-hydroxyaminocarbonyl” and “N-alkyl-N-hydroxyaminocarbonylalkyl”, denotes an amide group of the formula —C(═O)NH₂.

The terms “N-alkylaminocarbonyl” and “N,N-dialkylaminocarbonyl” denote aminocarbonyl radicals which have been substituted with one alkyl radical and with two alkyl radicals, respectively. More preferred are “lower alkylaminocarbonyl” having lower alkyl radicals as described above attached to an aminocarbonyl radical.

The terms “N-arylaminocarbonyl” and “N-alkyl-N-arylaminocarbonyl” denote aminocarbonyl radicals substituted, respectively, with one aryl radical, or one alkyl and one aryl radical.

The term “N-cycloalkylaminocarbonyl” denotes aminocarbonyl radicals which have been substituted with at least one cycloalkyl radical. More preferred are “lower cycloalkylaminocarbonyl” having lower cycloalkyl radicals of three to seven carbon atoms, attached to an aminocarbonyl radical.

The term “aminoalkyl” embraces alkyl radicals substituted with amino radicals.

The term “alkylaminoalkyl” embraces aminoalkyl radicals having the nitrogen atom substituted with an alkyl radical. Even more preferred are lower alkylaminoalkyl radicals having one to three carbon atoms.

The term “heterocyclylalkyl” embraces heterocyclic-substituted alkyl radicals. More preferred heterocyclylalkyl radicals are “5- or 6-membered heteroarylalkyl” radicals having alkyl portions of one to six carbon atoms and a 5- or 6-membered heteroaryl radical. Even more preferred are lower heteroarylalkyl radicals having alkyl portions of one to three carbon atoms. Examples include such radicals as pyridylmethyl and thienylmethyl.

The term “aralkyl” embraces aryl-substituted alkyl radicals. Preferable aralkyl radicals are “lower aralkyl” radicals having aryl radicals attached to alkyl radicals having one to six carbon atoms. Even more preferred are lower aralkyl radicals phenyl attached to alkyl portions having one to three carbon atoms. Examples of such radicals include benzyl, diphenylmethyl and phenylethyl. The aryl in the aralkyl may be additionally substituted with halo, alkyl, alkoxy, haloalkyl and haloalkoxy.

The term “arylalkenyl” embraces aryl-substituted alkenyl radicals. Preferable arylalkenyl radicals are “lower arylalkenyl” radicals having aryl radicals attached to alkenyl radicals having two to six carbon atoms. Examples of such radicals include phenylethenyl. The aryl in the arylalkenyl may be additionally substituted with halo, alkyl, alkoxy, haloalkyl and haloalkoxy.

The term “arylalkynyl” embraces aryl-substituted alkynyl radicals. Preferable arylalkynyl radicals are “lower arylalkynyl” radicals having aryl radicals attached to alkynyl radicals having two to six carbon atoms. Examples of such radicals include phenylethynyl. The aryl in the aralkynyl may be additionally substituted with halo, alkyl, alkoxy, haloalkyl and haloalkoxy.

The terms benzyl and phenylmethyl are interchangeable.

The term “alkylthio” embraces radicals containing a linear or branched alkyl radical, of one to ten carbon atoms, attached to a divalent sulfur atom. Even more preferred are lower alkylthio radicals having one to three carbon atoms. An example of “alkylthio” is methylthio, (CH₃—S—).

The term “haloalkylthio” embraces radicals containing a haloalkyl radical, of one to ten carbon atoms, attached to a divalent sulfur atom. Even more preferred are lower haloalkylthio radicals having one to three carbon atoms. An example of “haloalkylthio” is trifluoromethylthio.

The term “alkylsulfinyl” embraces radicals containing a linear or branched alkyl radical, of one to ten carbon atoms, attached to a divalent —S(═O)— atom. More preferred are lower alkylsulfinyl radicals having one to three carbon atoms.

The term “arylsulfinyl” embraces radicals containing an aryl radical, attached to a divalent —S(═O)— atom. Even more preferred are optionally substituted phenylsulfinyl radicals.

The term “haloalkylsulfinyl” embraces radicals containing a haloalkyl radical, of one to ten carbon atoms, attached to a divalent —S(═O)— atom. Even more preferred are lower haloalkylsulfinyl radicals having one to three carbon atoms.

The terms “N-alkylamino” and “N,N-dialkylamino” denote amino groups which have been substituted with one alkyl radical and with two alkyl radicals, respectively. More preferred alkylamino radicals are “lower alkylamino” radicals having one or two alkyl radicals of one to six carbon atoms, attached to a nitrogen atom. Even more preferred are lower alkylamino radicals having one to three carbon atoms. Suitable “alkylamino” may be mono or dialkylamino such as N-methylamino, N-ethylamino, N,N-dimethylamino, N,N-diethylamino or the like.

The term “arylamino” denotes amino groups which have been substituted with one or two aryl radicals, such as N-phenylamino. The “arylamino” radicals may be further substituted on the aryl ring portion of the radical.

The term “heteroarylamino” denotes amino groups which have been substituted with one or two heteroaryl radicals, such as N-thienylamino. The “heteroarylamino” radicals may be further substituted on the heteroaryl ring portion of the radical.

The term “aralkylamino” denotes amino groups which have been substituted with one or two aralkyl radicals. More preferred are phenyl-C₁-C₃-alkylamino radicals, such as N-benzylamino. The “aralkylamino” radicals may be further substituted on the aryl ring portion of the radical.

The terms “N-alkyl-N-arylamino” and “N-aralkyl-N-alkylamino” denote amino groups which have been substituted with one aralkyl and one alkyl radical, or one aryl and one alkyl radical, respectively, to an amino group.

The term “arylthio” embraces aryl radicals of six to ten carbon atoms, attached to a divalent sulfur atom. An example of “arylthio” is phenylthio.

The term “aralkylthio” embraces aralkyl radicals as described above, attached to a divalent sulfur atom. More preferred are phenyl-C₁-C₃-alkylthio radicals. An example of “aralkylthio” is benzylthio.

The term “aralkylsulfonyl” embraces aralkyl radicals as described above, attached to a divalent sulfonyl radical. More preferred are phenyl-C₁-C₃-alkylsulfonyl radicals.

The term “aryloxy” embraces optionally substituted aryl radicals, as defined above, attached to an oxygen atom. Examples of such radicals include phenoxy.

The term “aralkoxy” embraces oxy-containing aralkyl radicals attached through an oxygen atom to other radicals. More preferred aralkoxy radicals are “lower aralkoxy” radicals having optionally substituted phenyl radicals attached to lower alkoxy radical as described above.

For a compound of Formula II, groups R⁶ to R¹⁰, the following terms are defined:

“Alkyl”, “alkenyl,” and “alkynyl” unless otherwise noted are each straight chain or branched chain hydrocarbons of from one to twenty carbons for alkyl or two to twenty carbons for alkenyl and alkynyl in the present invention and therefore mean, for example, methyl, ethyl, propyl, butyl, pentyl or hexyl and ethenyl, propenyl, butenyl, pentenyl, or hexenyl and ethynyl, propynyl, butynyl, pentynyl, or hexynyl respectively and isomers thereof.

“Aryl” means a fully unsaturated mono- or multi-ring carbocycle, including, but not limited to, substituted or unsubstituted phenyl, naphthyl, or anthracenyl.

“Heterocycle” means a saturated or unsaturated mono- or multi-ring carbocycle wherein one or more carbon atoms can be replaced by N, S, P, or O. This includes, for example, the following structures: 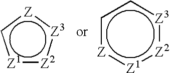
wherein Z, Z¹, Z² or Z³ is C, S, P, O, or N, with the proviso that one of Z, Z¹ Z² or Z³ is other than carbon, but is not O or S when attached to another Z atom by a double bond or when attached to another O or S atom. Furthermore, the optional substituents are understood to be attached to Z, Z¹, Z² or Z³ only if Z, Z¹, Z² or Z³ is C.

The term “heteroaryl” means a fully unsaturated heterocycle.

In either “heterocycle” or “heteroaryl,” the point of attachment to the molecule of interest can be at the heteroatom or elsewhere within the ring.

Illustrative examples of heterocycle and heteroaryl groups are provided above in the definition of terms used for Formulas I″, I′, and I.

The term “hydroxy” means a group having the structure —OH.

The term “halogen” or “halo” means a fluoro, chloro, bromo or iodo group.

The term “haloalkyl” means alkyl substituted with one or more halogens.

The term “cycloalkyl” means a mono- or multi-ringed carbocycle wherein each ring contains three to ten carbon atoms, and wherein any ring can contain one or more double or triple bonds. Examples include radicals such as cyclopropyl, cyclobutyl, cyclopentyl, cyclohexyl, cycloalkenyl, and cycloheptyl. The term “cycloalkyl” additionally encompasses spiro systems wherein the cycloalkyl ring has a carbon ring atom in common with the seven-membered heterocyclic ring of the benzothiepine.

The term “oxo” means a doubly bonded oxygen.

The term “cycloalkylidene” means a mono- or multi-ringed carbocycle wherein a carbon within the ring structure is doubly bonded to an atom which is not within the ring structures.

The term “nitro” means a group having the formula —NO₂.

The term “sulfo” means a sulfo group, —SO₃H, or its salts.

The term “thio” means a group having the formula —SH.

The term “sulfoalkyl” means an alkyl group to which a sulfonate group is bonded, wherein the alkyl is bonded to the molecule of interest.

The term “aminosulfonyl” means a group having the formula —SO₂NH₂.

The term “alkylthio” means a moiety containing an alkyl radical which is attached to an sulfur atom, such as a methylthio radical. The alkylthio moiety is bonded to the molecule of interest at the sulfur atom of the alkylthio. The term “aryloxy” a moiety containing an aryl radical which is attached to an oxygen atom, such as a phenoxy radical. The aryloxy moiety is bonded to the molecule of interest at the oxygen atom of the aryloxy.

The term “alkenyloxy” a moiety containing an alkenyl radical which is attached to an oxygen atom, such as a 3-propenyloxy radical. The alkenyloxy moiety is bonded to the molecule of interest at the oxygen atom of the alkenyloxy.

The term “arylalkyl” means an aryl-substituted alkyl radical such as benzyl. The term “alkylarylalkyl” means an arylalkyl radical that is substituted on the aryl group with one or more alkyl groups.

The term “amino” means a group having the structure —NH₂. Optionally the amino group can be substituted for example with one, two or three groups such as alkyl, alkenyl, alkynyl, aryl, and the like.

The term “cyano” means a group having the structure —CN.

The term “heterocyclylalkyl” means an alkyl radical that is substituted with one or more heterocycle groups.

The term “heteroarylalkyl” means an alkyl radical that is substituted with one or more heteroaryl groups.

The term “alkylheteroarylalkyl” means a heteroarylalkyl radical that is substituted with one or more alkyl groups.

The term “alkoxy” means a moiety containing an alkyl radical which is attached to an oxygen atom, such as a methoxy radical. The alkoxy moiety is bonded to the molecule of interest at the oxygen atom of the alkoxy. Examples of such radicals include methoxy, ethoxy, propoxy, iso-propoxy, butoxy and tert-butoxy.

The term “carboxy” means the carboxy group, —CO₂H, or its salts.

The term “carbonyl” means a carbon atom doubly bonded to an oxygen atom.

The term “carboxyalkyl” means an alkyl radical that is substituted with one or more carboxy groups. Preferable carboxyalkyl radicals are “lower carboxyalkyl” radicals having one or more carboxy groups attached to an alkyl radical having one to six carbon atoms.

The term “carboxyheterocycle” means a heterocycle radical that is substituted with one or more carboxy groups.

The term “carboxyheteroaryl” means a heteroaryl radical that is substituted with one or more carboxy groups.

The term “carboalkoxyalkyl” means an alkyl radical that is substituted with one or more alkoxycarbonyl groups. Preferable carboalkoxyalkyl radicals are “lower carboalkoxyalkyl” radicals having one or more alkoxycarbonyl groups attached to an alkyl radical having one to six carbon atoms.

The term “carboxyalkylamino” means an amino radical that is mono- or di-substituted with carboxyalkyl. Preferably, the carboxyalkyl substituent is a “lower carboxyalkyl” radical wherein the carboxy group is attached to an alkyl radical having one to six carbon atoms.

When used in terms that contain a combination of terms, for example “alkylaryl” or “arylalkyl,” the individual terms (e.g., alkyl, aryl) listed above have the meaning indicated above.

The compounds of Formulas I″, I′, I, and II, and the pharmaceutically acceptable salts thereof, are selective COX-2 inhibitors, which means that they are selective inhibitors of the COX-2 over COX-1. Preferably, the compounds of Formulas I″, I′, I, and II, and the pharmaceutically acceptable salts thereof, when assayed with COX-2 have IC₅₀ values of less than about 0.5 μM, and also have selectivity ratios of COX-2 inhibition over COX-1 inhibition of at least 50, and more preferably of at least 100. The COX-2 and COX-1 inhibitory activity is determined according to biological method “b. Assay for COX-1 and COX-2 Activity” of U.S. Pat. No. 6,077,850, column 169, beginning at line 15. The selectivity ratio is the IC₅₀ determined with COX-1 divided by the IC₅₀ ratio determined with COX-2, wherein each IC₅₀ is the concentration of a compound of Formulas I″, I′, I, or II, or a the pharmaceutically acceptable salt thereof, in micromolar that is needed to inhibit the enzyme being assayed by 50%.

The compounds of Formulas I″, I′, I, and II, and the pharmaceutically acceptable salts thereof, may be formulated for pharmaceutical use and administered to a mammal, including a human, to treat diseases such as arthritis and pain as described in U.S. Pat. No. 6,034,256; 6,077,850; 6,218,427; or 6,271,253 or U.S. patent application Ser. No. 10/801,446 or 10/801,429.

Another aspect of this invention is any one of the above or below methods for converting, wherein the component (b) is a (2S)- or (2R)-enantiomer of a substituted 2-trifluoromethyl-2H-chromene-3-carboxylic acid or ester derivative thereof, or a non-racemic mixture thereof.

Another aspect of this invention is any one of the above or below methods of the present invention, wherein the component (b) is a (2S)- or (2R)-enantiomer of a compound of Formula I″, I′, or I, wherein X is S or NR^{a}, or a non-racemic mixture thereof. Another aspect of this invention is any one of the above or below methods of the present invention, wherein the component (b) is a (2S)- or (2R)-enantiomer of a compound of Formula II, wherein X is S or NH, or a non-racemic mixture thereof.

Another aspect of this invention is any one of the above or below methods of the present invention, wherein the component (b) is a (2S)- or (2R)-enantiomer of a compound of Formula I″, I′, or I wherein X is O, or a non-racemic mixture thereof.

Another aspect of this invention is any one of the above or below methods of the present invention, wherein the component (b) is a (2S)- or (2R)-enantiomer of a compound of Formula II wherein X is O, or a non-racemic mixture thereof.

Another aspect of this invention is any one of the above or below methods for converting, wherein the component (b) is:
(R)-6-chloro-7-tert-butyl-2-trifluoromethyl-2H-chromene-3-carboxylic acid; or
(S)-6-chloro-7-tert-butyl-2-trifluoromethyl-2H-chromene-3-carboxylic acid; or the component (b) is a non-racemic mixture having a major component which is:
(S)-6-chloro-7-tert-butyl-2-trifluoromethyl-2H-chromene-3-carboxylic acid; and
a minor component which is the antipode (R)-6-chloro-7-tert-butyl-2-trifluoromethyl-2H-chromene-3-carboxylic acid.

Another aspect of this invention is any one of the above or below methods for converting, wherein the component (b) is:
(R)-6-chloro-8-methyl-2-trifluoromethyl-2H-chromene-3-carboxylic acid;
(S)-6-chloro-8-methyl-2-trifluoromethyl-2H-chromene-3-carboxylic acid;
(R)-6-chloro-5,7-dimethyl-2-trifluoromethyl-2H-chromene-3-carboxylic acid;
(S)-6-chloro-5,7-dimethyl-2-trifluoromethyl-2H-chromene-3-carboxylic acid;
(R)-6,8-dimethyl-2-trifluoromethyl-2H-chromene-3-carboxylic acid;
(S)-6,8-dimethyl-2-trifluoromethyl-2H-chromene-3-carboxylic acid;
(R)-8-ethyl-6-trifluoromethoxy-2-trifluoromethyl-2H-chromene-3-carboxylic acid; or
(S)-8-ethyl-6-trifluoromethoxy-2-trifluoromethyl-2H-chromene-3-carboxylic acid; or
the component (b) is a non-racemic mixture having a major component which is:
(S)-6-chloro-8-methyl-2-trifluoromethyl-2H-chromene-3-carboxylic acid;
(S)-6-chloro-5,7-dimethyl-2-trifluoromethyl-2H-chromene-3-carboxylic acid;
(S)-6,8-dimethyl-2-trifluoromethyl-2H-chromene-3-carboxylic acid; or
(S)-8-ethyl-6-trifluoromethoxy-2-trifluoromethyl-2H-chromene-3-carboxylic acid; and
a minor component which is the antipode:
(R)-6-chloro-8-methyl-2-trifluoromethyl-2H-chromene-3-carboxylic acid;
(R)-6-chloro-5,7-dimethyl-2-trifluoromethyl-2H-chromene-3-carboxylic acid;
(R)-6,8-dimethyl-2-trifluoromethyl-2H-chromene-3-carboxylic acid; or
(R)-8-ethyl-6-trifluoromethoxy-2-trifluoromethyl-2H-chromene-3-carboxylic acid, respectively.

Another aspect of this invention is any one of the above or below methods for converting, wherein component (b) is:
(S)-8-chloro-6-methoxy-2-trifluoromethyl-2H-chromene-3-carboxylic acid;
(R)-8-chloro-6-methoxy-2-trifluoromethyl-2H-chromene-3-carboxylic acid;
(S)-6-chloro-7-(1,1-dimethyl-2-hydroxyethyl)-2-trifluoromethyl-2H-chromene-3-carboxylic acid;
(R)-6-chloro-7-(1,1-dimethyl-2-hydroxyethyl)-2-trifluoromethyl-2H-chromene-3-carboxylic acid;
(S)-6-chloro-7-benzyl-2-trifluoromethyl-2H-chromene-3-carboxylic acid;
(R)-6-chloro-7-benzyl-2-trifluoromethyl-2H-chromene-3-carboxylic acid;
(S)-6-ethyl-8-methyl-2-trifluoromethyl-2H-chromene-3-carboxylic acid;
(R)-6-ethyl-8-methyl-2-trifluoromethyl-2H-chromene-3-carboxylic acid;
(S)-6-chloro-5-methyl-2-trifluoromethyl-2H-chromene-3-carboxylic acid;
(R)-6-chloro-5-methyl-2-trifluoromethyl-2H-chromene-3-carboxylic acid;
(S)-6,8-dichloro-7-cyclohexylmethoxy-2-trifluoromethyl-2H-chromene-3-carboxylic acid;
(R)-6,8-dichloro-7-cyclohexylmethoxy-2-trifluoromethyl-2H-chromene-3-carboxylic acid;
(S)-6-chloro-8-methyl-2-trifluoromethyl-2H-chromene-3-carboxylic acid;
(R)-6-chloro-8-methyl-2-trifluoromethyl-2H-chromene-3-carboxylic acid;
(S)-6-chloro-2-trifluoromethyl-1,2-dihydro-quinoline-3-carboxylic acid;
(R)-6-chloro-2-trifluoromethyl-1,2-dihydro-quinoline-3-carboxylic acid;
(S)-6-trifluoromethoxy-8-ethyl-2-trifluoromethyl-2H-chromene-3-carboxylic acid ethyl ester;
(R)-6-trifluoromethoxy-8-ethyl-2-trifluoromethyl-2H-chromene-3-carboxylic acid ethyl ester;
(S)-6,8-dimethyl-2-trifluoromethyl-2H-chromene-3-carboxylic acid ethyl ester;
(R)-6,8-dimethyl-2-trifluoromethyl-2H-chromene-3-carboxylic acid ethyl ester;
(S)-6-chloro-7-tert-butyl-2-trifluoromethyl-2H-chromene-3-carboxylic acid ethyl ester; or
(R)-6-chloro-7-tert-butyl-2-trifluoromethyl-2H-chromene-3-carboxylic acid ethyl ester; or
component (b) is a non-racemic mixture of:
(R)- and (S)-8-chloro-6-methoxy-2-trifluoromethyl-2H-chromene-3-carboxylic acid;
(R)- and (S)-6-chloro-7-(1,1-dimethyl-2-hydroxyethyl)-2-trifluoromethyl-2H-chromene-3-carboxylic acid;
(R)- and (S)-6-chloro-7-benzyl-2-trifluoromethyl-2H-chromene-3-carboxylic acid;
(R)- and (S)-6-ethyl-8-methyl-2-trifluoromethyl-2H-chromene-3-carboxylic acid;
(R)- and (S)-6-chloro-5-methyl-2-trifluoromethyl-2H-chromene-3-carboxylic acid;
(R)- and (S)-6,8-dichloro-7-cyclohexylmethoxy-2-trifluoromethyl-2H-chromene-3-carboxylic acid;
(R)-(S)-6-chloro-8-methyl-2-trifluoromethyl-2H-chromene-3-carboxylic acid;
(R)- and (S)-6-chloro-2-trifluoromethyl-1,2-dihydro-quinoline-3-carboxylic acid;
(R)- and (S)-6-trifluoromethoxy-8-ethyl-2-trifluoromethyl-2H-chromene-3-carboxylic acid ethyl ester;
(R)- and (S)-6,8-dimethyl-2-trifluoromethyl-2H-chromene-3-carboxylic acid ethyl ester; or
(R)- and (S)-6-chloro-7-tert-butyl-2-trifluoromethyl-2H-chromene-3-carboxylic acid ethyl ester.

Another aspect of this invention is any one of the above or below methods for converting, wherein R is NH₂.

Another aspect of this invention is any one of the above or below methods for converting, wherein R^{Q} is —(CH₂)_{q}NH₂ or —(CH₂)_{q}OH and q is 1.

Another aspect of this invention is any one of the above or below methods for converting, wherein R^{K} and R^{Q} are taken together to form a C₁-C₄ alkylene.

A method of the present invention may further comprise a preliminary step of subjecting any mixture of the (2S)- and (2R)-enantiomers of a substituted 2-trifluoromethyl-2H-chromene-3-carboxylic acid or derivative thereof, to enantioselective fractional crystallization with or without any chiral auxiliary or a preliminary step of subjecting the any mixture to enantioselective multicolumn chromatography, to yield a component (b), wherein the component (b) is then subjected to the method of converting step of the present invention as described herein.

Any method of the present invention may further comprise a subsequent step of subjecting the mixture of the (2S)- and (2R)-enantiomers that has been relatively enriched in the antipode of the (2S)- or (2R)-enantiomer to enantioselective fractional crystallization with or without a chiral auxiliary or a subsequent step of subjecting the mixture of the (2S)- and (2R)-enantiomers that has been relatively enriched in the antipode of the (2S)- or (2R)-enantiomer to enantioselective multicolumn chromatography.

Steady state recycling chromatography includes SSRC known by the trade name CYCLOJET® (Novasep Societe Par Actions Simplifiee, Pompey, France) and by the trademark “SteadyCycle™” (CYBA Technologies, LLC, Mystic, Conn., USA). Steady state recycling chromatography includes chromatography methods that use two columns or a single column.

The phrase “multicolumn chromatography” means a chromatography method that utilizes more than one column connected in series.

The (2S)- or (2R)-enantiomer may be separated from the compound of formula R^{K}N(H)—C(H)(R^{Q})CH₂—R^{L} and from the compound of formula M¹OR^{Y} or M²(OR^{Y})₂ by conventional means such as acid or base extraction.

The term “racemizing” means a process of reducing the enantiomeric excess of a (2S)- or (2R)-enantiomer of a substituted 2-trifluoromethyl-2H-chromene-3-carboxylic acid or derivative thereof. Racemizing may be performed on a single enantiomer or non-racemic mixture thereof and the process may or may not produce a racemic mixture of the enantiomers.

The method of the present invention can be repeated one or more times to maximize recovery yield of the antipode of the (2S)- or (2R)-enantiomer or the mixture that has been relatively enriched in the antipode of the (2S)- or (2R)-enantiomer.

In general, the method of converting step of the present invention works best when the reaction mixture is heated above room temperature (i.e., above 25° C.). For practical reasons, heating below a temperature of 300° C. is preferred. More preferred is heating to a temperature of the reaction mixture of from about 50° C. to about 250° C. More preferred is heating to a temperature of the reaction mixture of from about 100° C. to about 200° C. Also more preferred is heating to a temperature of the reaction mixture of from about 100° C. to about 150° C. or from about 150° C. to about 200° C.

After purification, if needed, to remove any impurities, the mixture that has been relatively enriched in the antipode of the (2S)- or (2R)-enantiomer will be suitable for an enantioselective multicolumn chromatography such as enantioselective steady state recycling chromatography or enantioselective simulated moving bed chromatography. Preferred is wherein the mixture that has been relatively enriched in the antipode of the (2S)- or (2R)-enantiomer is subjected to the multicolumn chromatography via a recycle stream or recycle/feed stream.

The mixture that has been relatively enriched in the antipode of the (2S)- or (2R)-enantiomer may be a racemic or non-racemic mixture.

A non-racemic mixture of enantiomers is any mixture other than a 50.0%:50.0% mixture of the enantiomers.

The (2S)-enantiomer of a substituted 2-trifluoromethyl-2H-chromene-3-carboxylic acid or derivative thereof is the antipode of the corresponding (2R)-enantiomer of the substituted 2-trifluoromethyl-2H-chromene-3-carboxylic acid or derivative thereof, respectively. The (2R)-enantiomer of a substituted 2-trifluoromethyl-2H-chromene-3-carboxylic acid or derivative thereof is the antipode of the corresponding (2S)-enantiomer of the substituted 2-trifluoromethyl-2H-chromene-3-carboxylic acid or derivative thereof, respectively.

Another aspect of this invention is any one of the above or below methods for converting, wherein the enantiomeric excess of the mixture that has been relatively enriched in the antipode of the (2S)- or (2R)-enantiomer is less than 80%, less than 70%, or less than 60% of the enantiomeric excess of the component (b).

Another aspect of this invention is any one of the above or below methods for converting, wherein the enantiomeric excess of the mixture that has been relatively enriched in the antipode of the (2S)- or (2R)-enantiomer is less than 50%, less than 40%, or less than 30% of the enantiomeric excess of the component (b).

A mixture produced by a method of converting of the present invention that has been relatively enriched in the antipode of the (2S)- or (2R)-enantiomer will have a lower e.e. than the e.e. of the component (b). As the amount of the antipode relative to the amount of the (2S)- or (2R)-enantiomer goes up during a method of converting of the present invention, the e.e. of the mixture produced by a method of converting will go down.

The e.e. values characterized by having an enantiomeric excess that is less than 90%, less than 80%, less than 70%, and the like are calculated as follows:
[100×(the e.e. of the mixture that has been relatively enriched in the antipode of the (2S)- or (2R)-enantiomer)]÷(the e.e. of the non-racemic mixture of a (2S)- and (2R)-enantiomer of the substituted 2-trifluoromethyl-2H-chromene-3-carboxylic acid or derivative thereof) is less than 90%, less than 80%, less than 70%, and the like, respectively.

For illustration, a mixture that has been relatively enriched in the antipode of the (2S)- or (2R)-enantiomer having an e.e. that is less than 90% of the e.e. of component (b), wherein the e.e. of component (b) was 95%, 54%, or 20%, means that the e.e. of the mixture that has been relatively enriched in the antipode of the (2S)- or (2R)-enantiomer is less than 85.5%, 48.6%, or 18%, respectively. Enantiomeric excess as used herein is determined using enantiomeric purity data that are obtained according to the method of Analytical Method (I) below.

Alternatively, the components (a), (b), and optionally (c) are dissolved, or partially dissolved and partially suspended, in a solvent, although the method of converting step of the present invention may be carried out without a solvent if upon heating the components form a solution or partial solution. The solvent mixture may be a mother liquor from an enantioselective fractional crystallization.

Solvents in which the method of converting step of the present invention may be carried out include polar solvents, nonpolar solvents, and buffered basic aqueous solutions, and mixtures thereof, provided that the boiling point of the solvent or solvent mixture is sufficiently high to allow heating of components to a temperature sufficient for carrying out the invention method.

Reaction mixtures include solutions and suspensions in the solvent or solvent mixture enantiomer(s). The solvent or solvent mixture may be a mother liquor from an enantioselective fractional crystallization.

Optionally, the components (a), (b), and optionally (c) are dissolved in a solvent, although method of converting step of the present invention may be carried out without a solvent if upon heating the components form a solution or partial solution (e.g., a melt). Alternatively, the components (b) and optionally (c) is/are dissolved, or partially dissolved and partially suspended, in the component (a).

Another aspect of this invention is any one of the above or below methods for converting, wherein the enantioselective multicolumn chromatography eluate stream contains a mobile phase which comprises:
a single polar solvent;
a solution comprising a polar solvent and an acidic solvent wherein the polar solvent is at least 99% volume/volume of the solution and the acidic solvent is less than 1% volume/volume of the solution; or
a solution comprising a polar solvent, an acidic solvent, and a nonpolar solvent wherein the polar solvent is less than or equal to 50% volume/volume of the mixture, the acidic solvent is less than 1% volume/volume of the solution and the nonpolar solvent is greater than 50% volume/volume of the solution. Preferred is the use of the above mobile phases with a substituted 2-trifluoromethyl-2H-chromene-3-carboxylic acid or ester derivative thereof.

Another aspect of this invention, the mobile phase comprises:
a buffered neutral aqueous solution and a polar solvent;
a buffered acidic aqueous solution and a polar solvent; or a buffered basic aqueous solution and a polar solvent,
   wherein the polar solvent comprises from about 5% to about 95% volume/volume of the mobile phase. Preferred is the use of the above mobile phases with a pharmaceutically acceptable salt of the substituted 2-trifluoromethyl-2H-chromene-3-carboxylic acid or ester derivative thereof.

The mobile phase may also comprise at least one additive. An additive suitable for chromatography of the acid or ester on a chiral stationary phase is typically an amine such as trimethylamine, triethylamine, and the like or an organic salt such as sodium or potassium acetate or an inorganic salt such as ammonium acetate or ammonium chloride. An additive suitable for chromatography of the salt of the acid or ester on a reverse phase, chiral stationary phase is typically an inorganic salt such as those described herein.

The solvents useful in the method of converting step or enantioselective multicolumn chromatography step of the present invention include polar solvents, nonpolar solvents, and buffered basic aqueous solutions, and mixtures thereof.

A polar solvent includes solvents that contain from 1 to 8 carbon atoms and 1 oxygen atom and is selected from straight or branched acyclic C₁-C₈ alcohols such as methanol, ethanol, propanol, iso-propyl alcohol, butanol, and the like, cyclic C₃-C₈ alcohols such as cyclopropanol, cyclobutanol, and the like, C₄-C₈ ethers such as ethyl ether, tert-butyl methyl ether, tetrahydrofuran, tetrahydropyran, and the like, straight or branched C₃-C₈ alkanones such as acetone, butanone, 2-pentanone, 3-pentanone, 3,3-dimethyl-2-pentanone, and the like, and C₃-C₈ cycloalkanones such as cyclopropanone, cyclobutanone, cyclopentanone, cyclohexanone, 3-methylcyclopentanone, and the like.

A polar solvent also includes solvents that contain from 1 to 8 carbon atoms and 2 oxygen atoms and is selected from supercritical fluid such as carbon dioxide, C₃-C₈ esters such as methyl acetate, ethyl acetate, propyl propionate, methyl butyrate, and the like, C₃-C₈ lactones such as beta-butyrolactone, gamma-butyrolactone, gamma-valerolactone, delta-valerolactone, and the like, and C₃-C₈ bis ethers such as 2-methoxy-ethyl ether, and the like.

A polar solvent also includes solvents that contain from 1 to 8 carbon atoms and 1 nitrogen atom and is selected from C₂-C₈ nitriles such as acetonitrile, propionitrile, butyronitrile, and the like.

A polar solvent also includes solvents that contain from 1 to 8 carbon atoms, 1 oxygen atom, and 1 nitrogen atom and is selected from C₂-C₈ carboxylic amides such as C₂-C₈ amides such as acetamide, N-methyl-acetamide, N,N-dimethylformamide, butyramide, and the like and C₄-C₈ lactams such as beta-lactam, 2-pyrrolidinone, 1-methyl-2-pyrrolidinone, delta-valerolactam, and the like.

A polar solvent also includes solvents that contain from 1 to 8 carbon atoms and 2 or 3 chlorine atoms and is selected from dichloro-(C₁-C₈ hydrocarbons) such as dichloromethane, and trichloro-(C₁-C₈ hydrocarbons) such as 1,1,1-trichloroethane, and the like.

A polar solvent also includes solvents selected from a C₃-C₆ alkanone such as acetone, a C₂-C₆ nitrile such as acetonitrile, and a C₁-C₆ alcohol such as methanol, ethanol, 1-propanol, 2-propanol, 1-butanol, 2-butanol, and the like.

A polar solvent may comprise from about 1% to about 99%, from about 5% to about 95%, from about 10% to about 90%, from about 20% to about 80%, or from about 30% to about 70% volume/volume of the mobile phase.

A polar solvent includes solvents such as ethanol, methanol, or acetonitrile.

A nonpolar solvent includes solvents that contain a straight chain or branched C₅-C₁₀ acyclic hydrocarbon comprises n-pentane, iso-pentane, n-hexane, n-heptane, 2,2,5-trimethylhexane, and the like.

A nonpolar solvent also includes solvents that contain a C₅-C₁₀ cyclic hydrocarbon comprises cyclopentane, cyclohexane, methylcyclopentane, cycloheptane, and the like.

A buffered basic aqueous solution comprises water, a salt such as a sodium or potassium perchlorate, biphosphate, phosphate, bisulfate, sulfate, and the like and a base selected from sodium acetate, potassium acetate, sodium hydroxide, potassium hydroxide, and the like.

Eluate from an enantioselective multicolumn chromatography may be collected for analysis of any material dissolved therein or for isolation and recovery of any material dissolved therein by conventional means such as by evaporation of mobile phase, optionally with crystallization of the material. Alternatively, eluate may be introduced into a converting unit followed by introduction of the resulting converted mixture of enantiomers to the stationary phase of the chromatography unit via a recycle stream.

An eluate stream from an enantioselective multicolumn chromatography means a raffinate stream, wherein the mobile phase contains dissolved therein a majority of one enantiomer of the acid, ester, or salt thereof, or an extract stream, wherein the mobile phase contains dissolved therein a majority of the other enantiomer of the acid, ester, or salt thereof.

The eluate can be monitored for the presence or absence of enantiomers of the substituted 2-trifluoromethyl-2H-chromene-3-carboxylic acid or derivative thereof by any conventional means such as, for example, by passing the eluate, or a portion thereof, through a detector. The detector may be compatible with liquid chromatography or not and may be capable of determining chirality or not. Illustrative examples of detectors compatible with liquid chromatography include ultraviolet detectors, photodiode array detectors that may scan ultraviolet light wavelengths from about 210 nm wavelength to about 320 nm wavelength (e.g., 210 nm, 240 nm, 254 nm, 280 nm, or 290 nm) to detect UV-active components, devices that monitor rotation of plane polarized light such as the IBZ CHIRALYSER available from JM Science, Inc., Grand Island, N.Y., refractive index detectors, and evaporative light scattering detectors.

Alternatively, eluate may be monitored by timing fractions (e.g., when the retention time of an enantiomer is known); by sampling untimed or timed fractions and analyzing the samples by, for example, visual inspection, UV light illumination in conjunction with visual inspection, non-enantioselective or enantioselective HPLC, nuclear magnetic resonance, mass spectrometry, derivatization and analysis of the resulting derivative, and the like; by evaporating fractions and analyzing the resulting residue for the presence of an enantiomer such as by visual inspection, UV light illumination in conjunction with visual inspection, melting point, non-enantioselective or enantioselective HPLC, nuclear magnetic resonance spectrometry, mass spectrometry, and the like; or by adding a derivatizing agent to fractions of the eluate or to the residue therefrom, and analyzing the resulting derivative as described above. Any method of monitoring that may be used to determine the presence of an enantiomer of the acids, esters, or pharmaceutically acceptable salts thereof, even if the method of monitoring cannot determine optical characteristics (i.e., the optical purity or ee of an enantiomer) of the enantiomer or whether the enantiomer is present with its antipode or not, is useful for monitoring the eluate.

Monitoring is any process or activity by which one of ordinary skill in the art would know whether any portion of eluate would contain, contains, or did contain at least one of the enantiomers.

In another aspect of the present invention, the compound of formula R^{K}N(H)—C(H)(R^{Q})CH₂—R^{L} is a chiral secondary amine and is also useful in an enantioselective fractional crystallization step of a method of the present invention.

In another aspect of the present invention, the compound of formula R^{K}N(H)—C(H)(R^{Q})CH₂—R^{L} is replaced in the method for converting step with a chiral secondary amine. A chiral secondary amine is capable of being used as component (a) instead of the compound of formula R^{K}N(H)—C(H)(R^{Q})CH₂—R^{L} according to a method of this invention and is also capable of forming a crystalline salt with a substituted 2-trifluoromethyl-2H-chromene-3-carboxylic acid or acid derivative thereof. The crystalline salt may or may not enable an enantioselective fractional crystallization of the (2S)- or (2R)-enantiomer. A chiral secondary amine useful in the method for converting includes N-methyl-L-tert-Leucinol, N-methyl-L-Valinol, L-Prolinol, and the like. Any chiral secondary amines recited below in the lists of chiral amine auxiliaries may be useful in the method for converting of the present invention.

All chiral amine auxiliaries are useful in the preliminary step or subsequent step to the method of converting step of the present invention. Such a chiral amine auxiliary may be selected from the group consisting of: N-methyl-L-tert-Leucinol, N-methyl-L-Valinol, L-Prolinol, L-tert-Leucinol, (+)-Cinchonine, (+)-Quinine, (1R,2S)-(+)-cis-1-Amino-2-indanol, (DHQ)₂ PHAL, L-Proline, L-Phenyl glycine methyl ester, (R)-N-Benzyl-1-(1-naphthy)ethylamine, Tetramisole HCl, (1S,2S)-(+)-Thiomicamine, R-(+)-4-Diphenylmethyl-2-oxazolidinone, R-(+)-N,N-Dimethyl-1-phenylethylamine, L-Valinol, (1R,2R)-(−)-1,2-Diaminocyclohexane, (1R,2S)-2-Amino-1,2-diphenylethanol, (+)-Bis [(R)-1-phenylethyl] Amine, L-Prolinol, (S)-(−)-a-Methyl-benzylamine, (1S,2S)-(+)-2-Amino-1-phenyl-1,3-propanediol, (1R,2S)-(−)-Ephedrine, L-Phenylalanine ethyl ester, L-Phenylalaninol, (R)-(−)-3-methyl-2-butylamine, (1R,2R)-(+)-1,2-Diphenyl ethylenediamine, (1S,2R)-(+)-Norephedrine, (R)-(+)-N-Benzyl-α-Methylbenzylamine, (+)-(2S,3R)-4-Dimethyl amino-3-methyl-1,2-diphenyl-2-butanol, R-(+)-1-(1-Naphthyl)ethylamine, R-(+)-1-(4-Bromophenyl)ethylamine, (−)-Cinchonidine, D-Glucamine, (S)-(−)-1-Benzyl-2-pyrrolidinemethanol, (1R,2S)-(−)-N-Methylephedrine, (+)-Quinidine, (R)-(−)-2-Phenylglycinol, R-(−)-1-(4-Nitrophenyl)ethylamine, R-(−)-2-Amino-1-butanol, (R)-(−)-1-Cyclohexylethylamine, N-Methyl-D-glucamine, (8S,9R)-(−)-N-Benzylcinchoninium chloride, 1-Deoxy-1-(methylamino)-D-galactitol, (1R,2S)-(+)-cis-[-2-(Benzylamine)cyclohexyl]methanol, (1R,2R)-(−)-2-Amino-1-(4-nitrophenyl)-1,3-propanediol, L-Phenylalanine methyl ester, (1S,2S)-(+)-Pseudoephedrine, and (S)-1-methoxy-2-propylamine. Also useful is a chiral auxiliary selected from the group consisting of the enantiomers of the above-recited compounds (e.g., a chiral auxiliary which is (R)-1-methoxy-2-propylamine).

Such a chiral amine auxiliary may also be selected from the group consisting of: (R)-(−)-1-Amino-2-propanol, (−)-cis-Myrtanylamine, (R)-1-(4-Methylphenyl)ethylamine, (S)-Aminotetraline, (R)-(−)-sec-butylamine, (R)-(−)-Tetrahydrofurfurylamine, (R)-3,3-dimethyl-2-butylamine, (R)-(−)-2-Aminoheptane, L-(+)-Isoleucinol, L-Leucinol, (R)-(−)-aminoindan, H-Methioninol, (S)-(−)-N,alpha-dimethyl-benzylamine, (S)-(−)-1-Phenylpropylamine, S-(−)-3-Tert-butylamino-1,2-propanediol, (R)-1-Methyl-3-phenylpropylamine, (R)-3-Amino-3-phenylpropan-1-ol, (R)-1-(3-methoxyphenyl)ethylamine, (R)-(+)-1(4-Methoxyphenyl)ethylamine, Methyl (R)-(+)-3-methyl glutarate, (S)-(−)-1-(2-Napthyl)ethylamine, L-tyrosinamide, S-Benzyl-L-cysteinol, (S)-1-phenyl-2-(p-tolyl)ethylamine, [R-(R*,R*)]-(+)-bis alpha-methylbenzylamine, (R)-(−)-N benzyl-2-phenylglycinol, L-tyrosinol, (R)-(+)-(3,4-dimethoxy)benzyl-1-phenylethylamine, and 1-deoxy-1-(octylamino)-D-glucitol. Also useful is a chiral auxiliary selected from the group consisting of the enantiomers of the above-recited compounds (e.g., a chiral auxiliary which is D-tyrosinol).

Such a chiral amine auxiliary may also be selected from the group consisting of: (S)-(−)-2-amino-3-phenyl-1-propanol, (R)-(+)-4-diphenylmethyl-2-oxozolidinone, (1R,2R)-(+)-1,2-diphenylethylenediamine, (+)-dehydroabietylamine, (+)-amphetamine, (+)-deoxyphedrine, and (+)-chloramphenicol intermediate. Also useful is a chiral auxiliary selected from the group consisting of the enantiomers of the above-recited compounds (e.g., a chiral auxiliary which is (−)-chloramphenicol intermediate).

The phrase “enantioselective fractional crystallization” includes any crystallization that enriches the e.e. of an enantiomer of a chiral compound, wherein the relatively enriched enantiomer is optionally in the crystal phase or in the mother liquor therefrom. Enantioselective fractional crystallizations include crystallizations from non-racemic mixtures of enantiomers without a chiral auxiliary and co-crystallizations from racemic and non-racemic mixtures with a chiral auxiliary. Enantioselective fractional crystallizations include a crystallization of the major or minor enantiomer component.

Optionally, the method of converting step of the present invention is an equilibrium process, which does not favor one enantiomer over its antipode, or a non-equilibrium process that facilitates formation of one enantiomer over its antipode. Typically a non-equilibrium process has at least one non-equilibrium step or, if there are no non-equilibrium steps, at least two steps in equilibrium.

With the present invention in mind, one of ordinary skill in the art can determine suitable parameters and conditions for converting a particular enantiomer to its antipode without undue experimentation.

The method of the present invention includes laboratory scale, preparative scale, and manufacturing scale methods.

The method of the present invention works whether the enantiomer is free of impurities or not, free of water or other solvates or not, is crystalline or amorphous, is liquid or solid, and the like.

### EXAMPLES

Representative examples of the method for converting are described below in Examples (I) to (III).

Enantiomeric excess for Examples (I) to (III) can be determined by enantioselective high-pressure liquid chromatography (“HPLC”) using the HPLC method described below in Analytical Method (I).

### Analytical Method (I)

Using a column with 0.46 cm inner diameter and 250 mm length filled with CHIRALPAK® AD™ (Chiral Technologies, Inc., Exton, Pa.) stationary phase, a 10 μL injection volume, by eluting at room temperature with mobile phase (volume proportions) 95%/5% heptane:ethanol with 0.1% trifluoroacetic acid, at room temperature, flow rate at 1 mL/minute isocratic, and detecting with a photodiode array detector at 254 nm wavelength, and a run time of from 10 to 40 minutes, enantiomeric purities can be determined. This method was not used to determine the e.e. values reported in the below Examples (I) to (II).

### Example (I)

In separate experiments, 100-mg of (R)-6,8-dichloro-2-trifluoromethyl-2H-chromene-3-carboxylic acid was combined with:
(a) H₂NCH₂CH₂OH (b.p. 170° C.);
(b) CH₃N(H)CH₂CH₂OH (b.p. 159° C.);
(c) (CH₃)₂NCH₂CH₂OH (b.p. 133° C.);
(d) CH₃CH₂OCH₂CH₂OH (b.p. 135° C.); or
(e) CH₃C(═O)OCH₂CH₂OH (b.p. 156° C.); the mixtures were refluxed for 4 hours, and allowed to cool. Enantioselective HPLC indicated that the e.e. of Example (I)(b) was 40.8%, the e.e. of Example (I)(c) was unaffected.

### Example (II)

In separate experiments, 100-mg of (R)-6,8-dichloro-2-trifluoromethyl-2H-chromene-3-carboxylic acid was combined with:
(a) CH₃N(H)CH₂CH₂OH (b.p. 159° C.);
(b) CH₃N(H)CH₂CH₂OH (b.p. 159° C.) with 1 mole equivalent of NaOH; or
(c) CH₃N(H)CH₂CH₂OH (b.p. 159° C.) with 2 mole equivalents of NaOH;
   the mixtures were refluxed for 4 hours, and allowed to cool. Enantioselective HPLC indicated that the e.e. values were as follows: Example (II)(a) e.e. was 21.9%, Example (II)(b) e.e. was 15.8%, and Example (II)(c) e.e. was 5.3%.

### Example (III)

The compound (R)-6,8-dichloro-2-trifluoromethyl-2H-chromene-3-carboxylic acid was combined with CH₃N(H)CH₂CH₂OH/water, 1:1, heated at 100° C. for 4 hours, and allowed to cool. No change in e.e. was observed by enantioselective HPLC.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. It is intended, therefore, that the invention be defined by the scope of the claims that follow and that such claims be interpreted as broadly as is reasonable.

All references cited above, including patents, patent applications, patent application publications, and scientific journals, are hereby incorporated herein by reference in their entireties and for all purposes.

What is claimed is:

1. A method for converting a (2S)- or (2R)-enantiomer of a substituted 2-trifluoromethyl-2H-chromene-3-carboxylic acid, or derivative thereof, the method comprising the step of:
Heating at a temperature of from greater than 30° C. to less than 300° C. a reaction mixture containing, but not limited to, components (a), (b), and optionally (c):
(a) A compound of formula R^{K}N(H)—C(H)(R^{Q})CH₂—R^{L};
(b) A (2S)- or (2R)-enantiomer of a substituted 2-trifluoromethyl-2H-chromene-3-carboxylic acid or derivative thereof; or
A non-racemic mixture having a major component which is a (2S)- or (2R)-enantiomer of the substituted 2-trifluoromethyl-2H-chromene-3-carboxylic acid or derivative thereof, and a minor component which is the antipode of the (2S)- or (2R)-enantiomer;
and optionally
(c) A compound of formula M¹OR^{Y} or M²(OR^{Y})₂;
to yield a mixture of the (2S)- and (2R)-enantiomers that has been relatively enriched in the antipode of the (2S)- or (2R)-enantiomer;
wherein the mixture that has been relatively enriched in the antipode of the (2S)- or (2R)-enantiomer is characterized as having an enantiomeric excess of the (2S)- or (2R)-enantiomer that is less than 90% of the enantiomeric excess of component (b);
wherein:
the temperature is the temperature of the reaction mixture;
M¹ is Na, K, or Cs;
M² is Mg, Zn, or Ca;
Each R^{Y} independently is hydrogen, C₁-C₄ alkyl, benzyl, or HSO₃⁻; or
Two R^{Y} are taken together to form SO₃⁻;
R^{K} independently is C₁-C₄ alkyl or benzyl;
R^{Q} is hydrogen, C₁-C₄ alkyl, phenyl, benzyl, 4-hydroxybenzyl, —(CH₂)_{q}NH₂, or —(CH₂)_{q}OH;
or R^{K} and R^{Q} are taken together to form a C₁-C₄ alkylene;
q is an integer of from 1 to 4;
R^{L} is OH or NH₂;
C₁-C₄ alkyl is an unsubstituted straight or branched chain hydrocarbon radical having from 1 to 4 carbon atoms;
C₁-C₄ alkylene is an unsubstituted straight or branched chain hydrocarbon diradical having from 1 to 4 carbon atoms; and wherein:
the substituted 2-trifluoromethyl-2H-chromene-3-carboxylic acid or derivative thereof, is a compound of Formulas I″, I′, I, or II 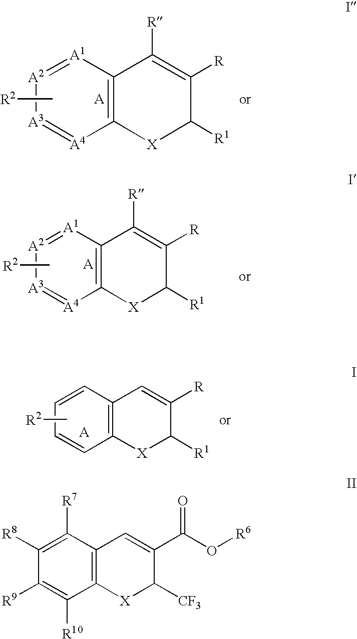
or a pharmaceutically acceptable salt thereof, wherein
for Formula I″:
wherein X is selected from O, S, and NR^{a};
wherein R^{a} is selected from hydrido, C₁-C₃-alkyl, (optionally substituted phenyl)-C₁-C₃-alkyl, acyl and carboxy-C₁-C₆-alkyl;
wherein R is selected from carboxyl, aminocarbonyl, C₁-C₆-alkylsulfonylaminocarbonyl and C₁-C₆-alkoxycarbonyl;
wherein R″ is selected from hydrido, phenyl, thienyl, C₁-C₆-alkyl and C₂-C₆-alkenyl;
wherein R¹ is CF₃;
wherein R² is one or more radicals independently selected from hydrido, halo, C₁-C₆-alkyl, C₂-C₆-alkenyl, C₂-C₆-alkynyl, halo-C₂-C₆-alkynyl, aryl-C₁-C₃-alkyl, aryl-C₂-C₆-alkynyl, aryl-C₂-C₆-alkenyl, C₁-C₆-alkoxy, methylenedioxy, C₁-C₆-alkylthio, C₁-C₆-alkylsulfinyl, aryloxy, arylthio, arylsulfinyl, heteroaryloxy, C₁-C₆-alkoxy-C₁-C₆-alkyl, aryl-C₁-C₆-alkyloxy, heteroaryl-C₁-C₆-alkyloxy, aryl-C₁-C₆-alkoxy-C₁-C₆-alkyl, C₁-C₆-haloalkyl, C₁-C₆-haloalkoxy, C₁-C₆-haloalkylthio, C₁-C₆-haloalkylsulfinyl, C₁-C₆-haloalkylsulfonyl, C₁-C₃-(haloalkyl-C₁-C₃-hydroxyalkyl, C₁-C₆-hydroxyalkyl, hydroxyimino-C₁-C₆-alkyl, C₁-C₆-alkylamino, arylamino, aryl-C₁-C₆-alkylamino, heteroarylamino, heteroaryl-C₁-C₆-alkylamino, nitro, cyano, amino, aminosulfonyl, C₁-C₆-alkylaminosulfonyl, arylaminosulfonyl, heteroarylaminosulfonyl, aryl-C₁-C₆-alkylaminosulfonyl, heteroaryl-C₁-C₆-alkylaminosulfonyl, heterocyclylsulfonyl, C₁-C₆-alkylsulfonyl, aryl-C₁-C₆-alkylsulfonyl, optionally substituted aryl, optionally substituted heteroaryl, aryl-C₁-C₆-alkylcarbonyl, heteroaryl-C₁-C₆-alkylcarbonyl, heteroarylcarbonyl, arylcarbonyl, aminocarbonyl, C₁-C₆-alkoxycarbonyl, formyl, C₁-C₆-haloalkylcarbonyl and C₁-C₆-alkylcarbonyl; and
wherein the A ring atoms A¹, A², A³ and A⁴ are independently selected from carbon and nitrogen with the proviso that at least two of A¹, A², A³ and A⁴ are carbon;
or wherein R² together with ring A forms a radical selected from naphthyl, quinolyl, isoquinolyl, quinolizinyl, quinoxalinyl and dibenzofuryl;
for Formula I′:
wherein X is selected from O, S, and NR^{a};
wherein R^{a} is selected from hydrido, C₁-C₃-alkyl, (optionally substituted phenyl)-C₁-C₃-alkyl, alkylsulfonyl, phenylsulfonyl, benzylsulfonyl, acyl and carboxy-C₁-C₆-alkyl;
wherein R is selected from carboxyl, aminocarbonyl, C₁-C₆-alkylsulfonylaminocarbonyl and C₁-C₆-alkoxycarbonyl;
wherein R” is selected from hydrido, phenyl, thienyl, C₂-C₆-alkynyl and C₂-C₆-alkenyl;
wherein R¹ is CF₃;
wherein R² is one or more radicals independently selected from hydrido, halo, C₁-C₆-alkyl, C₂-C₆-alkenyl, C₂-C₆-alkynyl, halo-C₂-C₆-alkynyl, aryl-C₁-C₃-alkyl, aryl-C₂-C₆-alkynyl, aryl-C₂-C₆-alkenyl, C₁-C₆-alkoxy, methylenedioxy, C₁-C₆-alkylthio, C₁-C₆-alkylsulfinyl, —O(CF₂)₂O—, aryloxy, arylthio, arylsulfinyl, heteroaryloxy, C₁-C₆-alkoxy-C₁-C₆-alkyl, aryl-C₁-C₆-alkyloxy, heteroaryl-C₁-C₆-alkyloxy, aryl-C₁-C₆-alkoxy-C₁-C₆-alkyl, C₁-C₆-haloalkyl, C₁-C₆-haloalkoxy, C₁-C₆-haloalkylthio, C₁-C₆-haloalkylsulfinyl, C₁-C₆-haloalkylsulfonyl, C₁-C₃-(haloalkyl-C₁-C₃-hydroxyalkyl, C₁-C₆-hydroxyalkyl, hydroxyimino-C₁-C₆-alkyl, C₁-C₆-alkylamino, arylamino, aryl-C₁-C₆-alkylamino, heteroarylamino, heteroaryl-C₁-C₆-alkylamino, nitro, cyano, amino, aminosulfonyl, C₁-C₆-alkylaminosulfonyl, arylaminosulfonyl, heteroarylaminosulfonyl, aryl-C₁-C₆-alkylaminosulfonyl, heteroaryl-C₁-C₆-alkylaminosulfonyl, heterocyclylsulfonyl, C₁-C₆-alkylsulfonyl, aryl-C₁-C₆-alkylsulfonyl, optionally substituted aryl, optionally substituted heteroaryl, aryl-C₁-C₆-alkylcarbonyl, heteroaryl-C₁-C₆-alkylcarbonyl, heteroarylcarbonyl, arylcarbonyl, aminocarbonyl, C₁-C₆-alkoxycarbonyl, formyl, C₁-C₆-haloalkylcarbonyl and C₁-C₆-alkylcarbonyl; and
wherein the A ring atoms A¹, A², A³ and A⁴ are independently selected from carbon and nitrogen with the proviso that at least two of A¹, A², A³ and A⁴ are carbon;
or wherein R² together with ring A forms a radical selected from naphthyl, quinolyl, isoquinolyl, quinolizinyl, quinoxalinyl and dibenzofuryl;
for Formula I:
wherein X is selected from O or S or NR^{a};
wherein R^{a} is alkyl;
wherein R is selected from carboxyl, aminocarbonyl, alkylsulfonylaminocarbonyl and alkoxycarbonyl;
wherein R¹ is CF₃; and
wherein R² is one or more radicals selected from hydrido, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, optionally substituted aryl, optionally substituted heteroaryl, aralkylcarbonyl, heteroarylcarbonyl, arylcarbonyl, aminocarbonyl, and alkylcarbonyl;
or wherein R² together with ring A forms a naphthyl radical;
for Formula II:
wherein X is selected from O, S, and NH;
wherein R is H or alkyl; and
wherein R⁷, R⁸, R⁹, and R¹⁰ independently are selected from H, alkenyl, alkoxy, alkoxyalkyl, alkoxycarbonylalkyl, alkyl, alkylamino, alkylcarbonyl, alkylheteroaryl, alkylsulfonylalkyl, alkylthio, alkynyl, aminocarbonylalkyl, aryl, arylalkenyl, arylalkoxy, arylalkyl, arylalkylamino, arylalkynyl, arylcarbonyl, aryloxy, cyano, dialkylamino, halo, haloalkoxy, haloalkyl, heteroaryl, heteroarylalkoxy, heteroarylcarbonyl, hydroxy and hydroxyalkyl;
wherein each of aryl, wherever it occurs, is independently substituted with one to five substituents selected from the group consisting of alkyl, alkoxy, alkylamino, cyano, halo, haloalkyl, hydroxy, and nitro.

2. The method as in claim 1, wherein the component (b) is a (2S)- or (2R)-enantiomer of a compound of Formula I″, I′, I, or II wherein X is O, or a non-racemic mixture thereof.

3. The method as in claim 1, wherein the component (b) is a (2S)- or (2R)-enantiomer of a compound of Formula II wherein X is O and R⁶ is H, or a non-racemic mixture thereof.

4. The method as in claim 1, wherein the component (b) is (R)-6-chloro-7-tert-butyl-2-trifluoromethyl-2H-chromene-3-carboxylic acid, or the component (b) is a non-racemic mixture having a major component which is (R)-6-chloro-7-tert-butyl-2-trifluoromethyl-2H-chromene-3-carboxylic acid and a minor component which is the antipode (S)-6-chloro-7-tert-butyl-2-trifluoromethyl-2H-chromene-3-carboxylic acid.

5. The method as in claim 1, wherein the component (b) is:
(R)-6-chloro-8-methyl-2-trifluoromethyl-2H-chromene-3-carboxylic acid;
(R)-6-chloro-5,7-dimethyl-2-trifluoromethyl-2H-chromene-3-carboxylic acid;
(R)-6,8-dimethyl-2-trifluoromethyl-2H-chromene-3-carboxylic acid; or
(R)-8-ethyl-6-trifluoromethoxy-2-trifluoromethyl-2H-chromene-3-carboxylic acid; or
the component (b) is a non-racemic mixture having a major component which is:
(R)-6-chloro-8-methyl-2-trifluoromethyl-2H-chromene-3-carboxylic acid;
(R)-6-chloro-5,7-dimethyl-2-trifluoromethyl-2H-chromene-3-carboxylic acid;
(R)-6,8-dimethyl-2-trifluoromethyl-2H-chromene-3-carboxylic acid; or
(R)-8-ethyl-6-trifluoromethoxy-2-trifluoromethyl-2H-chromene-3-carboxylic acid; and
a minor component which is the antipode:
(S)-6-chloro-8-methyl-2-trifluoromethyl-2H-chromene-3-carboxylic acid;
(S)-6-chloro-5,7-dimethyl-2-trifluoromethyl-2H-chromene-3-carboxylic acid;
(S)-6,8-dimethyl-2-trifluoromethyl-2H-chromene-3-carboxylic acid; or
(S)-8-ethyl-6-trifluoromethoxy-2-trifluoromethyl-2H-chromene-3-carboxylic acid, respectively.

6. The method as in claim 1, wherein the reaction mixture further contains a means for enantioselective fractional crystallization of the antipode of the (2S)- or (2R)-enantiomer.

7. The method as in claim 1, wherein the component (b) is:
(R)-6-chloro-7-tert-butyl-2-trifluoromethyl-2H-chromene-3-carboxylic acid (+)-cinchonine salt; or
(R)-6-chloro-7-tert-butyl-2-trifluoromethyl-2H-chromene-3-carboxylic acid D-phenylalaninol salt; or
the component (b) is a non-racemic mixture having a major component which is:
(R)-6-chloro-7-tert-butyl-2-trifluoromethyl-2H-chromene-3-carboxylic acid (+)-cinchonine salt; or
(R)-6-chloro-7-tert-butyl-2-trifluoromethyl-2H-chromene-3-carboxylic acid D-phenylalaninol salt; and
a minor component which is the antipode:
(S)-6-chloro-7-tert-butyl-2-trifluoromethyl-2H-chromene-3-carboxylic acid (+)-cinchonine salt; or
(S)-6-chloro-7-tert-butyl-2-trifluoromethyl-2H-chromene-3-carboxylic acid D-phenylalaninol salt, respectively.

8. The method as in claim 1, wherein the component (b) is:
(R)-6-chloro-8-methyl-2-trifluoromethyl-2H-chromene-3-carboxylic acid
(R)-(+)-N-benzyl-α-methylbenzylamine salt;
(R)-6-chloro-5,7-dimethyl-2-trifluoromethyl-2H-chromene-3-carboxylic acid (−)-cinchonine salt;
(R)-6,8-dimethyl-2-trifluoromethyl-2H-chromene-3-carboxylic acid (R)-(+)-N-benzyl-α-methylbenzylamine salt; or
(R)-8-ethyl-6-trifluoromethoxy-2-trifluoromethyl-2H-chromene-3-carboxylic acid (R)-(+)-N-benzyl-α-methylbenzylamine salt; or
the component (b) is a non-racemic mixture having a major component which is:
(R)-6-chloro-8-methyl-2-trifluoromethyl-2H-chromene-3-carboxylic acid,
(R)-(+)-N-benzyl-α-methylbenzylamine salt;
(R)-6-chloro-5,7-dimethyl-2-trifluoromethyl-2H-chromene-3-carboxylic acid, (−)-cinchonine salt;
(R)-6,8-dimethyl-2-trifluoromethyl-2H-chromene-3-carboxylic acid, (R)-(+)-N-benzyl-α-methylbenzylamine salt; or
(R)-8-ethyl-6-trifluoromethoxy-2-trifluoromethyl-2H-chromene-3-carboxylic acid, (R)-(+)-N-benzyl-α-methylbenzylamine salt; and
a minor component which is the antipode:
(S)-6-chloro-8-methyl-2-trifluoromethyl-2H-chromene-3-carboxylic acid,
(R)-(+)-N-benzyl-α-methylbenzylamine salt;
(S)-6-chloro-5,7-dimethyl-2-trifluoromethyl-2H-chromene-3-carboxylic acid, (−)-cinchonine salt;
(S)-6,8-dimethyl-2-trifluoromethyl-2H-chromene-3-carboxylic acid, (R)-(+)-N-benzyl-α-methylbenzylamine salt; or
(S)-8-ethyl-6-trifluoromethoxy-2-trifluoromethyl-2H-chromene-3-carboxylic acid, (R)-(+)-N-benzyl-α-methylbenzylamine salt, respectively.

9. The method as in claim 1, wherein R^{L} is OH.

10. The method as in claim 1, wherein each R^{K} independently is C₁-C₄ alkyl.

11. The method as in claim 1, wherein R^{Q} is hydrogen.

12. The method as in claim 1, wherein the compound of formula M¹OR^{Y} is a compound of formula NaOR^{Y}, wherein R^{Y} is as defined above.

13. The method as in claim 1, wherein each R^{Y} is hydrogen.

14. The method as in claim 1, wherein R^{Y} is HSO₃⁻ or two R^{Y} are taken together to form SO₃⁻.
